(19) United States
(12) Patent Application Publication
(10) Pub. No.: US 2023/0338490 A1
(43) Pub. Date: Oct. 26, 2023

(54) CANINE CANCER VACCINE

(71) Applicant: Arizona Board of Regents on behalf of Arizona State University, Scottsdale, AZ (US)

(72) Inventor: Stephen Albert Johnston, Tempe, AZ (US)

(21) Appl. No.: 18/331,475

(22) Filed: Jun. 8, 2023

Related U.S. Application Data
(63) Continuation of application No. PCT/US2021/062683, filed on Dec. 9, 2021.
(60) Provisional application No. 63/123,742, filed on Dec. 10, 2020.

(51) Int. Cl. A61K 39/00; C07K 14/47; C07K 14/535; A61P 35/00; A61K 48/00

(52) U.S. Cl. CPC A61K 39/0011; A61K 48/005; A61P 35/00; C07K 14/4748; C07K 14/535; A61K 2039/53; A61K 2039/552; A61K 2039/55516

(57) ABSTRACT

Provided herein are vaccine compositions for use in immunotherapy for canine cancers, and methods of canine cancer immunotherapy using said compositions. The compositions and methods provided herein include DNA vaccines having two plasmids that encode thirty-one peptide antigens, plus a plasmid that encodes canine GMCSF.

### CROSS-REFERENCE

This application is a continuation of International Application No. PCT/US2021/062683, filed on Dec. 9, 2021, which claims the benefit of U.S. Provisional Application No. 63/123,742, filed Dec. 10, 2020, each of which is incorporated herein by reference in its entirety.

### SEQUENCE LISTING

This application contains a Sequence Listing which has been submitted electronically in XML format and is hereby incorporated by reference in its entirety. Said XML copy, created on May 30, 2023, is named 2023-05-30 Sequence Listing - CALV031C1.xml and is 51.4 KB in size.

### FIELD

This application relates to cancer, and in particular cancer vaccines for canines and methods of treatment with such vaccines.

### BACKGROUND

Canine tumors share many characteristics with human tumors, especially that of arising spontaneously in immunocompetent hosts. Additionally, their size suggests a similar degree of genetic and phenotypic diversity, suggesting a likely similar diversity of potential neoantigens and therefore antigen-reactive T cells. They are thus an interesting and potentially very useful mode for the evaluation of novel preventative cancer immunotherapy approaches.

Preliminary evidence suggests a remarkable conservation of expression of a series of novel frameshift peptides that appear to arise as a consequence of transcriptional infidelity through microsatellites, mis-initiation of translation of exon 1, and mis-splicing of exons at a much higher rate in cancers than in normal tissues. Some of these neoantigens appear to be conserved across species (including human, mouse, and dog) and across tumor types, resulting in detectable immune responses in many tumor-bearing hosts, and demonstrate evidence of tumor delay/prevention in several murine models of cancer.

### SUMMARY

Accordingly, the present disclosure relates to compositions and vaccines, and methods for cancer immunotherapy in canines.

Some embodiments provided herein relate to canine cancer vaccines. In some embodiments, the vaccines prevent cancer in a subject. In some embodiments, the subject is a canine. In some embodiments, the vaccines include one or more peptides having the sequence according to one of SEQ ID NOs: 1-34 and/or a nucleic acid capable of expressing the one or more peptides and a pharmaceutically acceptable carrier. In some embodiments, the vaccines include an adjuvant.

In some embodiments, the vaccines include a nucleic acid capable of expressing canine GMCSF. In some embodiments, the canine GMCSF has the amino acid sequence according to SEQ ID NO: 39. In some embodiments, the nucleic acid capable of expressing canine GMCSF is the vector NTC9382R-MCS-GMCSF.

In some embodiments, the vaccine includes one or more vectors expressing the peptide according to SEQ ID NOs: 1-34. In some embodiments, the vaccine includes a first vector capable of expressing a peptide having an amino acid sequence as set forth in any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 26, and a second vector capable of expressing a peptide having an amino acid sequence as set forth in any one or more of SEQ ID NO: 23, SEQ ID NO: 24, SEQ ID NO: 25, SEQ ID NO: 27, SEQ ID NO: 28, SEQ ID NO: 29, SEQ ID NO: 30, SEQ ID NO: 31, SEQ ID NO: 32, SEQ ID NO: 33, or SEQ ID NO: 34.

In some embodiments, any one or more of the amino acid sequences disclosed herein, or nucleic acids encoding the amino acid sequences are separated by a peptide linker. In some embodiments, the linker comprises an amino acid sequence according to SEQ ID NO: 36. In some embodiments, the first vector is NTC9382R-MCS-VACCS I and the second vector is NTC9382R-MCS-VACCS II.

In some embodiments, the vaccines include a peptide component. In some embodiments, the peptide component includes isolated peptides having an amino acid sequence as set forth in any one of SEQ ID NO: 1, SEQ ID NO: 2, and SEQ ID NO: 4, SEQ ID NO: 5, SEQ ID NO: 6, and SEQ ID NO: 11, SEQ ID NO: 13, SEQ ID NO: 14, and SEQ ID NO: 15, SEQ ID NO: 17, SEQ ID NO: 20, and SEQ ID NO: 21, SEQ ID NO: 22, SEQ ID NO: 23, SEQ ID NO: 26, SEQ ID NO: 28, and SEQ ID NO: 29, SEQ ID NO: 30, SEQ ID NO: 32, SEQ ID NO: 33, or SEQ ID NO: 34.

Some embodiments provided herein relate to methods of treating and/or inhibiting cancer in a canine subject. In some embodiments, the methods include administering to the canine subject a vaccine as described in any embodiment provided herein. For example, the methods include administering the vaccines including one or more peptides having the sequence according to one of SEQ ID NOs: 1-34 and/or a nucleic acid capable of expressing the one or more peptides and a pharmaceutically acceptable carrier, adjuvant, GMCSF, linker, and/or a peptide component, as described herein.

Some embodiments provided herein relate to methods of preventing cancer in a canine subject. In some embodiments, the methods include administering to the canine subject a vaccine as described in any embodiment provided herein. For example, the methods include administering the vaccines including one or more peptides having the sequence according to one of SEQ ID NOs: 1-34 and/or a nucleic acid capable of expressing the one or more peptides and a pharmaceutically acceptable carrier, adjuvant, GMCSF, linker, and/or a peptide component, as described herein.

Some embodiments provided herein relate to methods of eliciting an immune response in a canine subject. In some embodiments, the methods include administering to the canine subject a vaccine as described in any embodiment provided herein. For example, the methods include administering the vaccines including one or more peptides having the sequence according to one of SEQ ID NOs: 1-34 and/or a nucleic acid capable of expressing the one or more peptides and a pharmaceutically acceptable carrier, adjuvant, GMCSF, linker, and/or a peptide component, as described herein.

Some embodiments provided herein relate to compositions. In some embodiments, the compositions include one or more peptides having the sequence according to one of SEQ ID NOs: 1-34 and/or a nucleic acid capable of expressing the one or more peptides. In some embodiments, the compositions include one or more vectors expressing the peptide according to SEQ ID NOs: 1-34. In some embodiments, the compositions include a first vector capable of expressing a peptide having an amino acid sequence as set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 26, and a second vector capable of expressing a peptide having an amino acid sequence as set forth in SEQ ID NO: 23, SEQ ID NO: 24, SEQ ID NO: 25, SEQ ID NO: 27, SEQ ID NO: 28, SEQ ID NO: 29, SEQ ID NO: 30, SEQ ID NO: 31, SEQ ID NO: 32, SEQ ID NO: 33, or SEQ ID NO: 34. In some embodiments, the amino acid sequences are separated by a peptide linker. In some embodiments, the linker includes an amino acid sequence as set forth in SEQ ID NO: 36. In some embodiments, the first vector is NTC9382R-MCS-VACCS I and the second vector is NTC9382R-MCS-VACCS II. In some embodiments, the compositions include a pharmaceutically acceptable carrier.

Some embodiments provided herein relate to compositions that include isolated peptides having an amino acid sequence as set forth in SEQ ID NO: 1, SEQ ID NO: 2, SEQ ID NO: 4, SEQ ID NO: 5, SEQ ID NO: 6, SEQ ID NO: 11, SEQ ID NO: 13, SEQ ID NO: 14, SEQ ID NO: 15, SEQ ID NO: 17, SEQ ID NO: 20, SEQ ID NO: 21, SEQ ID NO: 22, SEQ ID NO: 23, SEQ ID NO: 26, SEQ ID NO: 28, SEQ ID NO: 29, SEQ ID NO: 30, SEQ ID NO: 32, SEQ ID NO: 33, or SEQ ID NO: 34.

Some embodiments provided herein relate to kits that include the vaccines as described herein or the compositions as described herein. For example, the kits include the vaccines or compositions that include one or more peptides having the sequence according to one of SEQ ID NOs: 1-34 and/or a nucleic acid capable of expressing the one or more peptides and a pharmaceutically acceptable carrier, adjuvant, GMCSF, linker, and/or a peptide component, as described herein. In some embodiments, the kits include instructions for use.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIGS. 1A-1B show vector maps for VACC-I/II (FIG. 1A) and the GMCSF (FIG. 1B).

FIGS. 2A-2B show an overview of the IgG positive reactivity of each frameshift (FS) antigen in dog cancer patient (n=116) and normal dog (n=52) by FS peptide array in one batch of the assays (an 800 FSP array). X axis indicates difference sources of the FS antigen. The positive cut off value of each peptide was calculated as the mean reactivity in non-cancer subjects plus two-fold SD, and at least one positive peptide for a positive FS antigen.

FIG. 3 shows the positive rate distribution of each FS peptide was compared between cancer group and normal group of all FSPs, p-value<0.0001.

### DETAILED DESCRIPTION

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage, and have their ordinary meaning as understood in light of the specification. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: A Comprehensive Desk Reference, published by VCH Publishers, Inc., 1995 (ISBN 9780471185710); and other similar references. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

To facilitate review of the various embodiments of this disclosure, the following explanations of specific terms are provided, along with particular examples. The terms provided herein have their ordinary meaning as understood in light of the specification.

Adjuvant: A vehicle used to enhance antigenicity; such as a suspension of minerals (alum, aluminum hydroxide, aluminum phosphate) on which antigen is adsorbed; or water-in-oil emulsion in which antigen solution is emulsified in oil (MF-59, Freund’s incomplete adjuvant), sometimes with the inclusion of killed mycobacteria (Freund’s complete adjuvant) to further enhance antigenicity (inhibits degradation of antigen and/or causes influx of macrophages). Adjuvants also include immunostimulatory molecules, such as cytokines, costimulatory molecules, and for example, immunostimulatory DNA or RNA molecules, such as CpG oligonucleotides.

An adjuvant is a substance distinct from the antigen for which an immune response is desired. In several embodiments, an adjuvant enhances T cell activation by promoting the innate immune response leading to the accumulation and activation of other leukocytes (accessory cells) at the site of antigen exposure. Thus, adjuvants may enhance accessory cell expression of T cell- activating co-stimulators and cytokines and may also prolong the expression of peptide-MHC complexes on the surface of antigen-presenting cells.

Antibody: Immunoglobulin molecules and immunologically active portions of immunoglobulin molecules, such as molecules that contain an antigen binding site that specifically binds (immunoreacts with) an antigen.

A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544-546, 1989) which consists of a VH domain; (v) an isolated complementarity determining region (CDR); and (vi) a F(ab′)2 fragment, a bivalent fragment comprising two Fab fragments linked by a disulfide bridge at the hinge region.

Immunoglobulins and certain variants thereof are known and many have been prepared in recombinant cell culture (e.g., see U.S. Pat. No. 4,745,055; U.S. Pat. No. 4,444,487; WO 88/03565; EP 256,654; EP 120,694; EP 125,023; Falkner et al., Nature 298:286, 1982; Morrison, J. Immunol. 123:793, 1979; Morrison et al., Ann Rev. Immunol 2:239, 1984). Humanized antibodies and fully human antibodies are also known in the art.

Administration: The introduction of a composition or agent into a subject by a chosen route. Administration can be local or systemic. For example, if the chosen route is intravenous, the composition is administered by introducing the composition into a vein of the subject.

Agent: Any substance or any combination of substances that is useful for achieving an end or result; for example, a substance or combination of substances useful for inducing an immune response in a subject. Agents include peptides, (such as disclosed herein) proteins, nucleic acid molecules, compounds, small molecules, organic compounds, inorganic compounds, or other molecules of interest. An agent can include a therapeutic agent, a diagnostic agent or a pharmaceutical agent. In some embodiments, the agent is a polypeptide agent. The skilled artisan will understand that particular agents may be useful to achieve more than one result.

Animal: Living multi-cellular vertebrate organisms, a category that includes, for example, mammals and birds. The term mammal includes both human and non-human mammals. Similarly, the term “subject” includes both human and veterinary subjects. In some embodiments, the animal is a dog, and may include any breed or any age of dog.

Antigen: A compound, composition, or substance that can stimulate the production of antibodies or a T cell response in an animal, including polypeptides that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

Immunogenic polypeptides and immunogenic peptides are non-limiting examples of antigens. In some examples, antigens include polypeptides derived from a pathogen of interest, such as a virus.

Array: An arrangement of molecules, such as biological macromolecules (such as peptides), in addressable locations on or in a substrate. A “microarray” is an array that is miniaturized so as to require or be aided by microscopic examination for evaluation or analysis. The array of molecules (“features”) makes it possible to carry out a very large number of analyses on a sample at one time. Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples can be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the subject features in the array are arranged regularly, for instance in a Cartesian grid pattern, which can be correlated to address information by a computer.

Binding or stable binding: An association between two substances or molecules, such as the association of an antibody with a peptide. Binding can be detected by any procedure known to one skilled in the art, such as by physical or functional properties of the formed complexes, such as a target/antibody complex.

Clinical outcome: Refers to the health status of a patient following treatment for a disease or disorder, or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

Contacting: Placement in direct physical association, including solid or liquid forms.

Control: A “control” refers to a sample or standard used for comparison with an experimental sample, such as a tumor sample obtained from a subject, such as a canine subject with a particular type of cancer. The control can be a sample obtained from a healthy subject or a non-tumor tissue sample obtained from a subject diagnosed with a particular type of cancer. A control can also be a historical control or standard reference value or range of values (such as a previously tested control sample, such as a group of subjects with poor prognosis, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Diagnostic: Identifying the presence or nature of a pathologic condition. Diagnostic methods differ in their sensitivity and specificity. The “sensitivity” of a diagnostic assay is the percentage of diseased subjects who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” means predicting the probability of development (for example, severity) of a pathologic condition.

Effective amount: An amount of agent, such as an agent that is sufficient to generate a desired response, such as an immune response. In some examples, an “effective amount” is one that treats (including prophylaxis) one or more symptoms and/or underlying causes of any of a disorder or disease, for example to treat and/or prevent cancer in a subject, such as a canine subject. In one example, an effective amount is a therapeutically effective amount. In one example, an effective amount is an amount that prevents one or more signs or symptoms of a particular disease or condition from developing, such as one or more signs or symptoms associated with cancer.

Expression: Translation of a nucleic acid into a peptide and/or protein. Peptides and/or proteins may be expressed and remain intracellular, become a component of the cell surface membrane, or be secreted into the extracellular matrix or medium.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter. A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al, Methods in Enzymology 153:516-544, 1987). A polynucleotide can be inserted into an expression vector that contains a promoter sequence, which facilitates the efficient transcription of the inserted genetic sequence.

Frameshift: “Frameshift” refers to an alteration in the reading frame of a coding RNA. This can be the result of an insertion or deletion of nucleotides and the juxtaposition of two sequences not naturally together, e.g., mis-splicing of exons. In some embodiments, the altered frame results in premature termination of the protein, resulting in a C-terminal extension of non-natural protein sequence.

Host cells: Cells in which a vector, such as a viral vector or DNA vector, can be propagated and its nucleic acid sequences expressed. The cell may be prokaryotic or eukaryotic. The term also includes any progeny of the subject host cell. It is understood that all progeny may not be identical to the parental cell since there may be mutations that occur during replication. However, such progeny is included when the term “host cell” is used.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence. “Specifically hybridizable” and “specifically complementary” are terms that indicate a sufficient degree of complementarity such that stable and specific binding occurs between the oligonucleotide (or it’s analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, though wash times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, NY, 1989, chapters 9 and 11.

Immune response: A response of a cell of the immune system, such as a B cell, T cell, or monocyte, to a stimulus. In one embodiment, the response is specific for a particular antigen (an “antigen-specific response”). In some embodiments, an immune response is a T cell response, such as a CD8+ response. In another embodiment, the response is a B cell response, and results in the production of antigen-specific antibodies. As used herein, “stimulating an immune response” refers to promoting or enhancing the response of the cells of the immune system to a stimulus, such as an antigen.

Immunogenic peptide: A peptide which comprises an allele-specific motif or other sequence such that the peptide will bind an MHC molecule and induce a cytotoxic T lymphocyte (“CTL”) response, or a B cell response (e.g., antibody production) against the antigen from which the immunogenic peptide is derived.

Immunogenic composition: A composition that includes an immunogenic polypeptide or nucleic acid or viral vector encoding an immunogenic polypeptide that induces a measurable immune response (such as a CTL response or measurable B cell response) against the immunogenic polypeptide. For example, in several embodiments, an immunogenic composition includes a viral vector expressing an immunogenic polypeptide that induces an immune response to an epitope on the immunogenic polypeptide that is also contained on a polypeptide expressed by a viral pathogen. In one example an immunogenic composition includes a nucleic acid encoding an immunogenic polypeptide, such as a nucleic acid vector that can be used to express the polypeptide (and thus be used to elicit an immune response against this polypeptide or an epitope on the polypeptide). In several examples, the immunogenic composition includes one or more adjuvants.

Immunotherapy: A method of evoking an immune response against a virus based on its production of target antigens. Immunotherapy based on cell-mediated immune responses involves generating a cell-mediated response to cells that produce particular antigenic determinants, while immunotherapy based on humoral immune responses involves generating specific antibodies to virus that produce particular antigenic determinants. In several embodiments, immunotherapy includes administration of prime-boost vaccination to a subject.

Inhibiting or treating a disease: Inhibiting the full development of a disease or condition, for example cancer. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component (such as a nucleic acid molecule, peptide, protein, or organelle) has been substantially separated or purified away from other biological components in the cell of the organism in which the component naturally occurs, e.g., other chromosomal and extra-chromosomal DNA and RNA, proteins and/organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids, such as probes and primers.

Neoantigen: “Neoantigen” includes, but is not limited to, antigens that are encoded by tumor-specific mutated genes or alterations in the processing or reading of RNA. Neoantigens are highly specific for tumors, and hence, some embodiments provided herein relate to tumor vaccines targeting neoantigens that effectively induce tumor-specific T cells in patients without killing normal cells, thereby achieving effective immune therapy or prevention of tumors.

Nucleotide: “Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof, Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (e.g., A, T, G, C), this also includes an RNA sequence (e.g., A, U, G, C) in which “U” replaces “T.” “Nucleotide” includes, but is not limited to, a monomer that includes a base linked to a sugar, such as a pyrimidine, purine or synthetic analogs thereof, or a base linked to an amino acid, as in a peptide nucleic acid (PNA).

A nucleotide is one monomer in a polynucleotide. A nucleotide sequence refers to the sequence of bases in a polynucleotide. Conventional notation is used herein to describe nucleotide sequences: the left-hand end of a single- stranded nucleotide sequence is the 5 ‘-end; the left-hand direction of a double- stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′- end of the RNA transcript are referred to as “upstream sequences;” sequences on the DNA strand having the same sequence as the RNA and which are 3′ to the 3′ end of the coding RNA transcript are referred to as “downstream sequences.” “cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. “Recombinant nucleic acid” refers to a nucleic acid having nucleotide sequences that are not naturally joined together. This includes nucleic acid vectors comprising an amplified or assembled nucleic acid, which can be used to transform a suitable host cell. A host cell that comprises the recombinant nucleic acid is referred to as a “recombinant host cell.” The gene is then expressed in the recombinant host cell to produce, such as a “recombinant polypeptide.”

A recombinant nucleic acid may serve a non-coding function (such as a promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence. Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences and amino acids sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art and exemplary methods are given below.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutical agent: A chemical compound or composition capable of inducing a desired therapeutic or prophylactic effect when properly administered to a subject or a cell. In some examples a pharmaceutical agent includes one or more of the disclosed polypeptides.

Pharmaceutically acceptable carriers: The pharmaceutically acceptable carriers of use are conventional. Remington’s Pharmaceutical Sciences, by E.W. Martin, Mack Publishing Co., Easton, PA, 19th Edition, 1995, describes compositions and formulations suitable for pharmaceutical delivery of the compositions disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Peptide: Any chain of amino acids, regardless of length or post-translational modification (e.g., glycosylation or phosphorylation). Immunogenic peptides include synthetic embodiments of peptides described herein. In addition, analogs (non-peptide organic molecules), derivatives (chemically functionalized peptide molecules obtained starting with the disclosed peptide sequences) and variants (homologs) of these proteins can be utilized in the methods described herein. Each polypeptide of this disclosure is comprised of a sequence of amino acids, which may be either L- and/or D- amino acids, naturally occurring and otherwise.

Peptides can be modified by a variety of chemical techniques to produce derivatives having essentially the same activity as the unmodified peptides, and optionally having other desirable properties. For example, carboxylic acid groups of the protein, whether carboxyl-terminal or side chain, can be provided in the form of a salt of a pharmaceutically-acceptable cation or esterified to form a C1-C16 ester, or converted to an amide of formula NR1R2 wherein R1 and R2 are each independently H or C1-C16 alkyl, or combined to form a heterocyclic ring, such as a 5- or 6- membered ring. Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to C1-C16 alkyl or dialkyl amino or further converted to an amide.

Hydroxyl groups of the peptide side chains may be converted to C1-C16 alkoxy or to a C1-C16 ester using well-recognized techniques. Phenyl and phenolic rings of the peptide side chains may be substituted with one or more halogen atoms, such as fluorine, chlorine, bromine or iodine, or with C1-C16 alkyl, C1-C16 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of an immunogenic Brachyury polypeptide having measurable or enhanced ability to generate an immune response. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, IL, pp. 165-174 and Principles of Pharmacology, Munson (ed.) 1995, Ch. 102, for descriptions of techniques used in CADD. Also included are mimetics prepared using such techniques.

Prime-boost vaccination: An immunotherapy including administration of a first immunogenic composition (the primer vaccine) followed by administration of a second immunogenic composition (the booster vaccine) to a subject to induce an immune response.

The booster vaccine is administered to the subject after the primer vaccine; the skilled artisan will understand a suitable time interval between administration of the primer vaccine and the booster vaccine, and examples of such timeframes are disclosed herein.

In some embodiments, the primer vaccine, the booster vaccine, or both primer vaccine and the booster vaccine additionally include an adjuvant.

Purified: The term purified does not require absolute purity; rather, it is intended as a relative term. Thus, for example, a purified polypeptide preparation is one in which the peptide or protein is more enriched than the peptide or protein is in its natural environment within a cell. In one embodiment, a preparation is purified such that the protein or peptide represents at least 50% of the total peptide or protein content of the preparation.

Self Antigen: Self antigens are derived from natural proteins. Because they are recognized as self by the immune system, they produce no or weak responses in an individual due to tolerance.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Within the context of an immunogenic peptide, a “conserved residue” is one which appears in a significantly higher frequency than would be expected by random distribution at a particular position in a peptide. In one embodiment, a conserved residue is one where the MHC structure may provide a contact point with the immunogenic peptide.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least 75%, for example at least 80%, sequence identity counted over the full-length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Tumor: All neoplastic cell growth and proliferation, whether malignant or benign, and all pre-cancerous and cancerous cells and tissues.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Vaccine: A preparation of immunogenic material capable of stimulating an immune response, administered for the prevention, amelioration, or treatment of infectious or other types of disease, such as cancer. The immunogenic material may include antigenic proteins, peptides or DNA derived from them.

The immunogenic material for a cancer vaccine may include, for example, a protein or peptide expressed by a tumor or cancer cell. Vaccines may elicit both prophylactic (preventative) and therapeutic responses. Methods of administration vary according to the vaccine, but may include inoculation, ingestion, inhalation or other forms of administration.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. Recombinant DNA vectors are vectors having recombinant DNA.

Recombinant RNA vectors are vectors having recombinant RNA. A vector can include nucleic acid sequences that permit it to replicate in a host cell, such as an origin of replication. A vector can also include one or more selectable marker genes and other genetic elements known in the art. Viral vectors are recombinant vectors having at least some nucleic acid sequences derived from one or more viruses.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting.

Some embodiments provided herein relate to high-density dog frameshift (FS) peptide arrays (FSP arrays). In some embodiments, the arrays include about 400,000 FS peptides. Such arrays may be used, for example, to analyze cancers in subjects, such as in dogs. For example, the arrays provided herein have been used to analyze 54 stage I dog cancers mainly from 5 cancer types and 54 non-cancers on this FSP array. The sera from dogs with 8 different, major types of cancer were screened in arrays containing the frameshift peptides arising from RNA mis-processing. Peptides that were in common in reactivity across multiple individuals and tumor types were selected. Some of these that had mouse homologs were tested in tumor models. Based on these studies a vaccine was constructed which elicits an immune response to anticipate any tumor that may arise. These vaccine components may also have clinical value as therapeutic vaccines. There are a set of FSP antigens that can confer protection against multiple cancer types across multiple individuals. These antigens can be used to prepare a prophylactic vaccine for multiple cancers. A list of FS antigens that offer protection in different mouse tumor models have been identified. Seven FS antigens have homologue FS antigens from the RNA error in both dog and human gene and were included in the vaccine. Thirteen of them that had positive IgG reactive in a set of 116 late-stage dog cancer sera from nine major cancer types on an 800 FS peptide array. These twenty FS antigens above have been tested in mouse and dogs for immunogenicity and safety analysis.

Disclosed herein are canine cancer vaccines that can be administered to dogs to prevent the occurrence of a number of cancer types. The disclosed vaccine is composed of frameshift peptide (FSP) neoantigens that are commonly produced in a number of disparate cancer types or nucleic acids encoding the same. The inventors have identified 36 FSP neoantigens that are commonly produced in dogs with cancer and these FSPs are immunogenic as measured by antibody reactivity. As discussed in the Examples below, a vaccine and a prime-boost vaccination protocol have been developed that include a DNA vaccine, including the prime portion of the vaccine that delivers DNA which encodes the FSP neoantigens, and a peptide vaccine, including the boost portion that delivers 20 FSP peptides in a single pool.

In some embodiments, a canine cancer vaccine includes one or more peptides having the sequence according to one of SEQ ID NOs: 1-34 and/or a nucleic acid capable of expressing the one or more peptides and a pharmaceutically acceptable carrier. The canine cancer vaccine maybe separated into its constituent components, such as one or more nucleic acid components, and/or peptide components, for example as part of a prime/boost vaccine strategy. In certain examples, the vaccine further includes a nucleic acid capable of expressing canine granulocyte-macrophage colony-stimulating factor (GMCSF), for example a vector, such as the vector NTC9382R-MCS-GMCSF. In certain embodiments the canine GMCSF has the amino acid sequence at least 95%, such as 95%, 96%, 97%, 98%, 99% or 100% identical to the amino acid sequence according to SEQ ID NO: 39.

In some embodiments, the vaccine incudes one or more vectors expressing the peptide scouring to SEQ ID NOs: 1-34. In some embodiments, the vaccine includes a first vector capable of expressing a peptide including the amino acid sequences according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 26, and a second vector capable of expressing a peptide including the amino acid sequences according to SEQ ID NO: 23, SEQ ID NO: 24, SEQ ID NO: 25, SEQ ID NO: 27, SEQ ID NO: 28, SEQ ID NO: 29, SEQ ID NO: 30, SEQ ID NO: 31, SEQ ID NO: 32, SEQ ID NO: 33, and SEQ ID NO: 34. In some embodiments, the amino acid sequences encoded by the nucleic acids are separated by a peptide linker. Peptide linkers are known in the art and include for example poly Gly-Ser. In some embodiments, the linker includes an amino acid sequence according to SEQ ID NO: 36. In some embodiments, the first vector is NTC9382R-MCS-VACCS I and the second vector is NTC9382R-MCS-VACCS II.

In some embodiments, the vaccine includes a peptide component, for example as part of a prime-boost protocol, such as where the nucleic acid component is given first and followed at some time later with the peptide component. In some embodiments, the peptide component includes isolated peptides with the amino acid sequences according to SEQ ID NO: 1, SEQ ID NO: 2, SEQ ID NO: 4, SEQ ID NO: 5, SEQ ID NO: 6, SEQ ID NO: 11, SEQ ID NO: 13, SEQ ID NO: 14, SEQ ID NO: 15, SEQ ID NO: 17, SEQ ID NO: 20, SEQ ID NO: 21, SEQ ID NO: 22, SEQ ID NO: 23, SEQ ID NO: 26, SEQ ID NO: 28, SEQ ID NO: 29, SEQ ID NO: 30, SEQ ID NO: 32, SEQ ID NO: 33, and SEQ ID NO: 34.

The compositions also can be formulated to contain an adjuvant in order to enhance the immunological response. Suitable adjuvants include, but are not limited to, mineral gels such as aluminum hydroxide, surface active substances such as lysolecithin, pluronic polyols, polyanions, other peptides, oil emulsions, and potentially useful human adjuvants such as Bacillus Calmette Guerin (BCG) and Corynebacterium parvum. Adjuvants for inclusion in the inventive composition desirably are safe, well tolerated, such as QS-21, Detox-PC, MPL-SE, MoGM-CSF, TiterMax-G, CRL-1005, GERBU, TERamide, PSC97B, Adjumer, PG-026, GSK-1, GcMAF, B-alethine, MPC-026, Adjuvax, CpG ODN, Betafectin, Alum, and MF59 (as described in, e.g., Kim et al., Vaccine, 18: 597 (2000)). Other adjuvants that can be administered to a mammal include lectins, growth factors, cytokines, and lymphokines (e.g., alpha-interferon, gamma-interferon, platelet derived growth factor (PDGF), gCSF, gMCSF, TNF, epidermal growth factor (EGF), IL- 1, IL-2, IL-4, IL-6, IL-8, IL-10, and IL- 12).ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund’s adjuvant, Freund’s complete, Freund’s incomplete (IF A), GM-CSF, GMDP, Gamma Inulin, Glycerol, HBSS (Hank’s Balanced Salt Solution), polyinosinic-polycytidylic acid stabilized with polylysine and carboxymethylcellulose (poly-ICLC, also referred to herein as Hiltonol), Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non- ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan. Checkpoint inhibitors can also be used. Some such checkpoint inhibitors are selected from the group consisting of a PD-1 inhibitor, a PD-L1 inhibitor, and a CTLA-4 inhibitor.

### Polynucleotides Encoding Canine Cancer Antigens

Polynucleotides encoding the antigenic peptide disclosed herein are provided. These polynucleotides include DNA, cDNA and RNA sequences which encode the antigen.

Methods for the manipulation and insertion of the nucleic acids of this disclosure into vectors are well known in the art (see for example, Sambrook et al., Molecular Cloning, a Laboratory Manual, 2d edition, Cold Spring Harbor Press, Cold Spring Harbor, N.Y., 1989, and Ausubel et al., Current Protocols in Molecular Biology, Greene Publishing Associates and John Wiley & Sons, New York, N.Y., 1994).

A nucleic acid encoding an antigenic peptide can be cloned or amplified by in vitro methods, such as the polymerase chain reaction (PCR), the ligase chain reaction (LCR), the transcription-based amplification system (TAS), the self- sustained sequence replication system (3SR) and the QP replicase amplification system (QB). For example, a polynucleotide encoding the protein can be isolated by polymerase chain reaction of cDNA using primers based on the DNA sequence of the molecule. A wide variety of cloning and in vitro amplification methodologies are well known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

The polynucleotides encoding an antigen include a recombinant DNA which is incorporated into a vector into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

DNA sequences encoding the antigen can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Polynucleotide sequences encoding antigens can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (such as ATG) in front of a protein-encoding gene, splicing signal for introns, maintenance of the correct reading frame of that gene to permit proper translation of mRNA, and stop codons.

Hosts can include microbial, yeast, insect and mammalian organisms.

Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as canine cells). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although cell lines may be used, such as cells designed to provide higher expression, desirable glycosylation patterns, or other features.

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding an antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

In some embodiments, a nucleic acid molecule that encodes an antigenic peptide is a nucleic acid provided herein as one that encodes any one of SEQ ID NOs: 1-34. In some embodiments, a nucleic acid molecule that encodes an antigen comprises a nucleic acid sequence at least about 95% identical, such as about 95%, about 96%, about 97%, about 98%, about 99% or even 100% identical to the nucleic acid sequence encoding any one of SEQ ID NOs: 1-34. In some embodiments, a nucleic acid molecule that encodes an antigen consists of a nucleic acid sequence encoding any one of SEQ ID NOs: 1-34.

### Vectors

The nucleic acid molecules encoding the antigenic peptides disclosed herein can be included in a vector, for example for expression of the antigen in a host cell, or for immunization of a subject as disclosed herein. In some embodiments, the vectors are administered to a subject as part of a prime-boost vaccination. In several embodiments, the vectors are included in a vaccine, such as a booster vaccine for use in a prime-boost vaccination. In embodiments a vector is VACC-II. In embodiments a vector is VACC-II.

### Therapeutic Methods and Pharmaceutical Compositions

Disclosed are methods of treating, inhibiting, and/or preventing cancer in a subject, such as a canine subject, for example by inducing an immune response, such as a protective immune response in a subject. In some embodiments, the disclosed methods include administering to the subject a vaccine including one or more of the immunogenic peptides disclosed herein, for example as isolated peptides and/or nucleic acids encoding the peptides. In some embodiments, the disclosed methods include administering to the subject a vaccine including a nucleic acid encoding one or more of the immunogenic peptides disclosed herein. In some embodiments, the disclosed methods include administering to the subject a vaccine including one or more of the immunogenic peptides disclosed herein and a vaccine including a nucleic acid encoding one or more of the immunogenic peptides disclosed herein. In some examples the vaccination including administering a priming vaccine and then, after a period of time has passed, administering to the subject a boosting vaccine, for example a peptide vaccine followed by a nucleic acid vaccine. The immune response is “primed” upon administration of the priming vaccine, and is “boosted” upon administration of the boosting vaccine.

The booster vaccine is administered to the subject after the primer vaccine. Administration of the priming vaccine and the boosting vaccine can be separated by any suitable timeframe. For example, the booster vaccine can be administered at least 1 week (e.g., 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 24 weeks, 28 weeks, 35 weeks, 40 weeks, 50 weeks, or at least 52 weeks, or a range defined by any two of the foregoing values) following administration of the first immunogenic compound. In some embodiments, the booster vaccine can be administered at about 1 week, 2 weeks 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 24 weeks, 28 weeks, 35 weeks, 40 weeks, 50 weeks, or about 52 weeks, or a range defined by any two of the foregoing values, following administration of the first immunogenic compound. More than one dose of priming vaccine and/or boosting vaccine can be provided in any suitable timeframe. The dose of the priming vaccine and boosting vaccine administered to the mammal depends on a number of factors, including the extent of any side-effects, the particular route of administration, and the like.

The methods can include selecting a subject in need of treatment, such as a canine subject at risk of cancer.

In embodiments a vaccine, such as a single vaccine or a prime and boost vaccine are typically administered as a pharmaceutically acceptable (e.g., physiologically acceptable) composition, which comprises a carrier, preferably a pharmaceutically carrier (e.g., physiologically acceptable). The vaccines can be administered alone, or in combination with at least one additional immunogenic agent or composition. It will be understood by those of skill in the art that the ability to produce an immune response after exposure to an antigen is a function of complex cellular and humoral processes, and that different subjects have varying capacity to respond to an immunological stimulus. Accordingly, the compositions disclosed herein are capable of eliciting an immune response in an immunocompetent subject, that is a subject that is physiologically capable of responding to an immunological stimulus by the production of a substantially normal immune response, e.g., including the production of antibodies that specifically interact with the immunological stimulus, and/or the production of functional T-cells (CD4⁺ and/or CD8⁺ T-cells) that bear receptors that specifically interact with the immunological stimulus.

Suitable formulations for the compositions include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. The compositions can be formulated to protect the nucleic acid sequence or vector from damage prior to administration. For example, the pharmaceutical composition can be formulated to reduce loss of the nucleic acid or construct on devices used to prepare, store, or administer the composition, such as glassware, syringes, or needles. The composition can be formulated to decrease the light sensitivity and/or temperature sensitivity of the nucleic acid sequence or construct. To this end, the composition preferably comprises a pharmaceutically acceptable liquid carrier, such as, for example, those described above, and a stabilizing agent selected from the group consisting of Polysorbate 80, L-arginine, polyvinylpyrrolidone, trehalose, and combinations thereof. Use of such a composition will extend the shelf life of the nucleic acid sequence or construct, facilitate administration, and increase the efficiency of the inventive method.

A composition also can be formulated to enhance transduction efficiency of the nucleic acid molecule or construct. In addition, one of ordinary skill in the art will appreciate that the composition can comprise other therapeutic or biologically-active agents. For example, factors that control inflammation, such as ibuprofen or steroids, can be part of the composition to reduce swelling and inflammation associated with in vivo administration of the composition. Antibiotics, e.g., microbicides and fungicides, can be present to treat existing infection and/or reduce the risk of future infection, such as infection associated with gene transfer procedures.

The composition also can be formulated to contain an adjuvant in order to enhance the immunological response. Suitable adjuvants include, but are not limited to, lysolecithin, pluronic polyols, polyanions, other peptides, oil emulsions, and potentially useful human adjuvants such as Bacillus Calmette Guerin (BCG) and Corynebacterium parvum. Adjuvants for inclusion in the inventive composition desirably are safe, well tolerated, such as QS-21, Detox-PC, MPL-SE, MoGM-CSF, TiterMax-G, CRL-1005, GERBU, TERamide, PSC97B, Adjumer, PG-026, GSK-1, GcMAF, B-alethine, MPC-026, Adjuvax, CpG ODN, Betafectin, Alum, and MF59 (as described in, e.g., Kim et al., Vaccine, 18: 597 (2000)). Other adjuvants that can be administered to a mammal include lectins, growth factors, cytokines, and lymphokines (e.g., alpha-interferon, gamma-interferon, platelet derived growth factor (PDGF), gCSF, gMCSF, TNF, epidermal growth factor (EGF), IL- 1, IL-2, IL-4, IL-6, IL-8, IL-10, and IL- 12).ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund’s adjuvant, Freund’s complete, Freund’s incomplete (IF A), GM-CSF, GMDP, Gamma Inulin, Glycerol, HBSS (Hank’s Balanced Salt Solution), polyinosinic-polycytidylic acid stabilized with polylysine and carboxymethylcellulose (poly-ICLC, also referred to herein as Hiltonol), Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non- ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan.

Any route of administration can be used to deliver the composition to the mammal. Indeed, although more than one route can be used to administer the composition, a particular route can provide a more immediate and more effective reaction than another route. In some examples, the composition is administered via intramuscular injection, for example, using a syringe or needleless delivery device. In some embodiments, the composition is administered by a gene gun. In this respect, this disclosure also provides a syringe or a needleless delivery device comprising the composition. The pharmaceutical composition also can be applied or instilled into body cavities, absorbed through the skin (e.g., via a transdermal patch), inhaled, ingested, topically applied to tissue, or administered parenterally via, for instance, intravenous, peritoneal, or intraarterial administration.

The composition can be administered in or on a device that allows controlled or sustained release, such as a sponge, biocompatible meshwork, mechanical reservoir, or mechanical implant. Implants (see, e.g., U.S. Pat. 5,443,505), devices (see, e.g., U.S. Pat. 4,863,457), such as an implantable device, e.g., a mechanical reservoir or an implant or a device comprised of a polymeric composition, are particularly useful for administration of the composition. The composition also can be administered in the form of a sustained-release formulation (see, e.g., U.S. Pat. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the composition administered will depend on a number of factors, including the size of a target tissue, the extent of any side-effects, the particular route of administration, and the like. The dose ideally comprises an “effective amount” of the composition, e.g., a dose of composition, which provokes a desired immune response in the mammal. The desired immune response can entail production of antibodies, protection upon subsequent challenge, immune tolerance, immune cell activation, and the like. One dose or multiple doses of the composition can be administered to a mammal to elicit an immune response with desired characteristics, including the production of specific antibodies, or the production of functional T-cells.

In some embodiments, the method includes administering a treatment to the subject, thereby eliciting an immune response and treating the tumor in the subject in need thereof.

Methods can include any known treatment used to control tumor growth, size, metastasis or other desired tumor activity or characteristic. In some examples, treatment can include radiation, surgical removal or administration of a composition such as a composition including one or more antibodies.

Effective dosages and schedules for administering the compositions may be determined empirically, and making such determinations is within the skill in the art. The dosage ranges for the administration of the compositions are those large enough to produce the desired effect in which the symptoms/disorder are/is affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter-indications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 mg/kg to up to 100 mg/kg of body weight or more per day, depending on the factors mentioned above.

Some embodiments provided herein relate to methods of making any of the vaccines as set forth herein.

### Kits

Kits are also provided. For example, kits for inhibiting and/or treating cancer in a canine viral subject. In several embodiments, the kit includes one or more containers including immunogenic compositions disclosed herein. In some embodiments, the kits further include a device for administration of the compositions disclosed herein. Such devices may include, for example, an injection device, a syringe, a needle-free injector, gene gun, an autoinjector, or any other device capable of administering the compositions provided herein.

In one embodiment, a kit includes instructional materials disclosing means of use for a vaccination as described herein. The instructional materials may be written, in an electronic form (such as a computer diskette or compact disk) or may be visual (such as video files). The kits may also include additional components to facilitate the particular application for which the kit is designed. Thus, for example, the kit may additionally include buffers and other reagents routinely used for the practice of a particular method. Such kits and appropriate contents are well known to those of skill in the art.

### Cancer and Tumor Type

In some embodiments, the cancer and/or tumor type comprises any cancer type that occurs in canines, including cancer types in humans that develop or are capable of developing in canines, such as, for example, Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt’s lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman’s Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing’s sarcoma, Extracranial Germ Cell Tumor, Extragonadal Germ Cell Tumor, Extrahepatic Bile Duct Cancer, Extramammary Paget’s disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin’s lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi’s sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget’s disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter’s transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom’s macroglobulinemia, Warthin’s tumor, or Wilms’ tumor.

The following examples are provided to illustrate particular features of certain embodiments. However, the particular features described below should not be construed as limitations on the scope of the disclosure, but rather as examples from which equivalents will be recognized by those of ordinary skill in the art.

### EXAMPLES

### Example 1: Preventative/Therapeutic Cancer Vaccine for Canine Subjects

Canine tumors share many characteristics with human tumors, especially that of arising spontaneously in immunocompetent hosts. Additionally, their size suggests a similar degree of genetic and phenotypic diversity, suggesting a likely similar diversity of potential neoantigens and therefore antigen-reactive T cells. They are thus an interesting and potentially very useful mode for the evaluation of novel preventative cancer immunotherapy approaches. The potential market for a dog preventative cancer vaccine is very large.

Preliminary evidence suggests a remarkable conservation of expression of a series of novel frameshift peptides that appear to arise as a consequence of transcriptional infidelity through microsatellites, mis-initiation of translation, and mis-splicing of exons at a much higher rate in cancers than in normal tissues. Some of these neoantigens appear to be conserved across species (including human, mouse and dog) and across tumor types, resulting in detectable immune responses in many tumor-bearing hosts, and demonstrate evidence of tumor delay/prevention in several murine models of cancer. Furthermore, pilot studies utilizing these vaccine antigens in normal laboratory dogs reveal measurable humoral and cell-mediated immune responses and absence of adverse effects following vaccination.

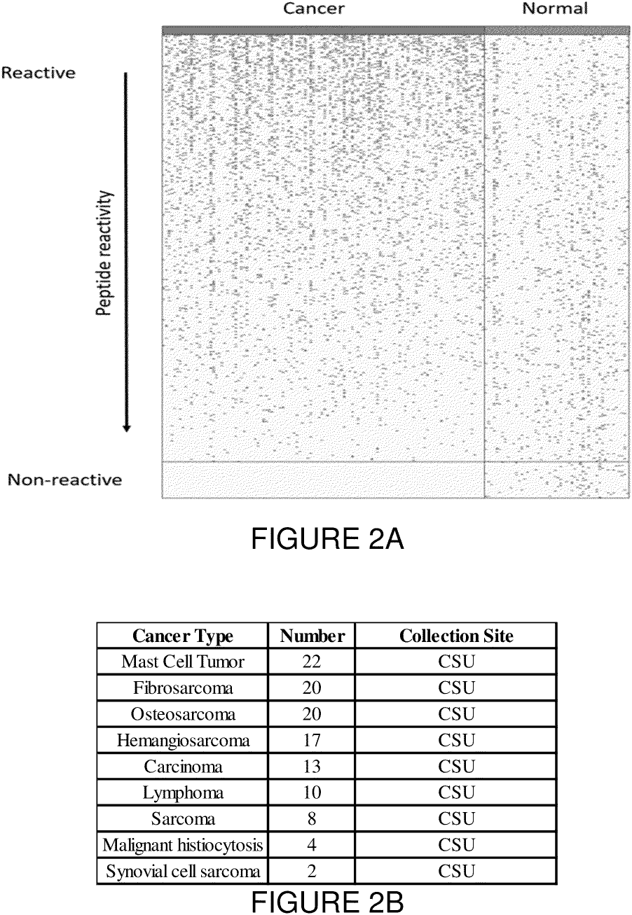

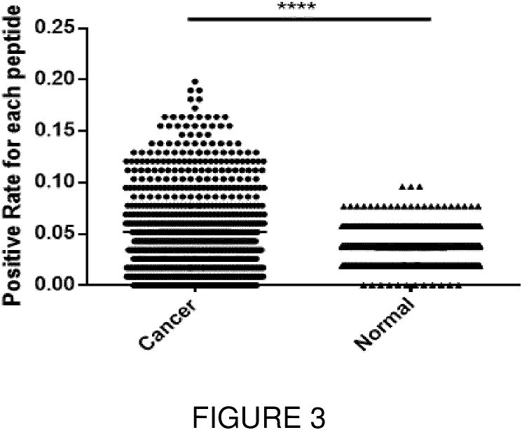

The sera from dogs with eight different, major types of cancer (see FIGS. 2A-2B and 3) were screened in arrays containing the frameshift peptides arising from RNA mis-processing. Peptides that were in common in reactivity across multiple individuals and tumor types were selected. Some of these that had mouse homologs were tested in mouse tumor models. Based on these studies a vaccine was constructed which elicit an immune response to anticipate any tumor that may arise. These vaccine components also have clinical value as therapeutic vaccines.

In this example the DNA vaccine includes 2 plasmids that encode 31 peptide antigens plus a third plasmid that encodes canine GMCSF. All plasmids in the DNA vaccine were constructed on the NTC9385R-MCS plasmid.

NTC9382R-LS-rCOMP-TT-VACCS I and II: These two plasmids encode a total of 31 selected antigens for the cancer vaccine (see below). Each antigen is linked by a repeat linker “GSGSGSGSGS” (SEQ ID NO: 36). These linked antigens are fused to the LS-rCOMP-TT peptide. Table 1 and Table 2 lists the amino acid sequence and peptide ID for the peptides encoded in each vaccine plasmid for the peptide vaccine.

NTC9382R-dog GMCSF: It encodes the dog GMCSF protein (NCBI reference sequence NP_001003245.1). It works as the adjuvant of the DNA vaccine.

TABLE 1

The amino acid sequences of the peptides encoded in the plasmids are shown in the table below

| ID | Sequence | SEQ ID NO: |
|---|---|---|
| VACCS-1 | TAIIGPNGSGCCRIYCHEEPQREDSSI | 1 |
| VACCS-2 | IPRMQPQASANPCQLLKPMVA | 2 |
| VACCS-3 | AVLLMCQMYQP WMCEEYYRLL | 3 |
| VACCS-4 | GRVIECDVVKGS CQDGEAVHRKPSPGGYRA | 4 |
| VACCS-5 | GEAVHRKPSPGGYRAGDSLTLRAVWEGAGM | 5 |
| VACCS-6 | QSRIPSKKCSLRKSTKSYKS CTSLKSLVPT | 6 |
| VACCS-7 | LLCVVSGKFVVSRSTFRHTGCYS | 7 |
| VACCS-8 | ILLNLSSESCTF | 8 |
| VACCS-9 | QWSSVTSLEWTAQERGCSSWLMKRTWMKSW | 9 |
| VACCS-10 | MKSWSLRDPSCRSTQEYISTQALWTPISTA | 10 |
| VACCS-11 | SPVASRGWVDTLKTPPSTTTGCPSWSPSWG | 11 |
| VACCS-12 | FLACVWTHSSSVSWKTWRGTTARRTGPTTC | 12 |
| VACCS-13 | SSAVAVGGPTPRIGPTPSRSSSPTGRAWS | 13 |
| VACCS-14 | NLDLQHQKKR KKKKDKVETQNRSQRANSTE | 14 |
| VACCS-15 | KMGKRKSLKRRKLQELPKRRKLRKNSNLSH | 15 |
| VACCS-16 | SHMMIKFFWMEMRYLCGSMTQFTLKHLSWD | 16 |
| VACCS-17 | NSPNGS RKRKKS CNFRINHGGLEPN | 17 |
| VACCS-18 | TQLHFAAESIIIIQLWKALAHSSCHLGA | 18 |
| VACCS-19 | KEKYRSRFYSKCLISIRSWIKTT | 19 |
| VACCS-20 | NLRRMVRGFIPCWIGTYIYLPKRKNLSYKR | 20 |
| VACCS-21 | QTSRWTRKGTISLSPLLIMFIKFRKFRSPR | 21 |
| VACCS-22 | ISPRVPCSLQKTMHLKYGLFSTFCLRTSIH | 22 |
| VACCS-23 | PRRRLLRWATYMSTSPTPLPRCATTPSTCA | 23 |
| VACCS-24 | WTLLASRCNATGFLRMVEVVEVEVEVGADQ | 24 |
| VACCS-25 | LTAKFKMGLKVFVSSTPEKADRVARLLLNL | 25 |
| VACCS-26 | MMRTKRLNKMASNQKI KLIRPRPKFRLASVDT | 26 |
| VACCS-27 | GWELGSVERMVRLQSRVRAFADSGVHSATP | 27 |
| VACCS-28 | YLAYREEGEVPELAVHGLAGDAVPRPQTAG | 28 |
| VACCS-29 | SSRESWEPGGKQQWFHKVPGSELVAPCWRL | 29 |
| VACCS-30 | RDRDGDHGARQAPRVGSQHLCAGTEHYGRR | 30 |
| VACCS-31 | GLGLFLEIFWPADP VPREWDSHREVAWPSR | 31 |
| VACCS-32 | PAGDVAGVVVGHGVRHPHRPPGLPQVWKGK | 32 |
| VACCS-34 | PPGGRGGLPPSPLLRVLYETWEDASSSPGN | 33 |
| VACCS-37 | FIARTISGITSRPMTPTKRPFTVLSAVRIT | 34 |
| VACCS-con | DLNQRGTELRSPSVDLNKPGRHSEPAAATG | 35 |

### Encoded Sequence of Each Plasmid

NTC9382R-LS-rCOMP-TT-VACCS-I:

MPSSVSWGILLLAGLCCLVPVSLAEDLNQRGTELRSPSVDLNKPGRHSEP
AAATGDLAPQMLRELQETNAALQDVRELLRQQVKEITFLKNTVMECDACG
DDPGGSILMQYIKANSKFIGITELKKLGGSNDIFNNFTVSFWLRVPKVSA
SHLEQYGGGSGDLTAIIGPNGSGCCRIYCHEEPQREDSSIGSGSGSGSGS
IPRMQPQASANPCQLLKPMVAGSGSGSGSGSAVLLMCQMYQPWMCEEYYR
LLGSGSGSGSGSGRVIECDVVKGSCQDGEAVHRKPSPGGYRAGDSLTLRA
VWEGAGMGSGSGSGSGSQSRIPSKKCSLRKSTKSYKSCTSLKSLVPTGSG
SGSGSGSLLCVVSGKFVVSRSTFRHTGCYSGSGSGSGSGSILLNLSSESC
TFGSGSGSGSGSQWSSVTSLEWTAQERGCSSWLMKRTWMKSWSLRDPSCR
STQEYISTQALWTPISTAGSGSGSGSGSSPVASRGWVDTLKTPPSTTTGC
PSWSPSWGPMSSPAASSVFLACVWTHSSSVSWKTWRGTTARRTGPTTCPK
PFSRSGSGSGSGSGSSSAVAVGGPTPRIGPTPSRSSSPTGRAWSGSGSGS
GSGSNLDLQHQKKRKKKKDKVETQNRSQRANSTEHQGEHNRAVGSGSGSG
SGSKMGKRKSLKRRKLQELPKRRKLRKNSNLSHMMIKFFWMEMRYLCGSM
TQFTLKHLSWDGSGSGSGSGSNSPNGSRKRKKSCNFRINHGGLEPNGSGS
GSGSGSTQLHFAAESIIIIQLWKALAHSSCHLGAGSGSGSGSGSKEKYRS
RFYSKCLISIRSWIKTTGSGSGSGSGSNLRRMVRGFIPCWIGTYIYLPKR
KNLSYKRQTSRWTRKGTISLSPLLIMFIKFRKFRSPRSSTLWSQCWPFFI
ASSFPTVGSGSGSGSGSISPRVPCSLQKTMHLKYGLFSTFCLRTSIHGSG
SGSGSGSMMRTKRLNKMASNQKIKLFRPRPKFRLASVDT (SEQ ID NO
: 37).

NTC9382R-LS-rCOMP-TT-VACCS-II:

MPSSVSWGILLLAGLCCLVPVSLAEDLNQRGTELRSPSVDLNKPGRHSEP
AAATGDLAPQMLRELQETNAALQDVRELLRQQVKEITFLKNTVMECDACG
DDPGGSILMQYIKANSKFIGITELKKLGGSNDIFNNFTVSFWLRVPKVSA
SHLEQYGGGSGDLLLSLLLQASLPMCCDGAGRSCPLEASGKKVWTLLASR
CNATGFLRMVEVVEVEVEVGADQLFPPRRRLLRWATYMSTSPTPLPRCAT
TPSTSLVRRKRWLFPQSRRQGAQNEGVLGHNHQEGSGSGSGSGSLTAKFK
MGLKVFVSSTPEKADRVARLLLNLNQKMKSNWPGSGSGSGSGSGWELGSV
ERMVRLQSRVRAFADSGVHSATPKERGQILGSGSGSGSGSYLAYREEGEV
PELAVHGLAGDAVPRPQTAGHPDPRKPGKRAHLLLPGSGSGSGSGSSSRE
SWEPGGKQQWFHKVPGSELVAPCWRLGAVSDPALQGSGSGSGSGSRDRDG
DHGARQAPRVGSQHLCAGTEHYGRREECRCHGLGECAVEQTLPRYGVPRP
GQPRGSGSGSGSGSGLGLFLEIFWPADPVPREWDSHREVAWPSRRGLTDH
FELWRWSRREQDTDIPSHGPGGSGSGSGSGSQAALQKDQLPTILLCLQPA
GDVAGVVVGHGVRHPHRPPGLPQVWKGKCHCGSEPQVGSGSGSGSGSDHQ
TPLVLYQEHSCRQHPAPPVTVLTELQERTLLQSSLWRISYKSLGSGSGSG
SGSGLSHTAGEAAARTPRPGCKGCPPGGRGGLPPSPLLRVLYETWEDASS
SPGNGSGSGSGSGSEGNWERQSPVWLLSQHGMVYLHSGHGCLLWYSRLRD
DKGSGSGSGSGSAPSNPSPTTLRVEGWRCTSWEASVTTGSCHKNLLTSFL
VNLTDKRRTFTGSGSGSGSGSWPPTQPRNPTSVCTVIRCFIARTICGITC
RPMTPTKRPFTVLSAVRITIRS (SEQ ID NO: 38).

NTC9382R-dog-GMCSF:

MWLQNLLFLGTVVCSISAPTRSPTLVTRPSQHVDAIQEALSLLNNSNDVT
AVMNKAVKVVSEVFDPEGPTCLETRLQLYKEGLQGSLTSLKNPLTMMANH
YKQHCPPTPESPCATQNINFKSFKENLKDFLFNIPFDCWKPVKK (SEQ
ID NO: 39).

### I. Peptide Vaccine

In this example the peptide boost vaccine includes a pool of 20 frameshift peptides administered separately from a Hiltonol (Poly-IC:LC) adjuvant. Peptides were synthesized and are formulated into a pool in aqueous buffer which is vialed and injected into subject canines. The sequence of the peptides included in the peptide boost are listed in the Table 2.

TABLE 2

| ID | Peptide Sequence | SEQ ID NO: |
|---|---|---|
| VACCS-1p | TAIIGPNGSGSSRIYSHEEPQREDSSI | 1 |
| VACCS-2p | IPRMQPQASANPSQLLKPMVA | 2 |
| VACCS-4p | GRVIESDVVKGSSQDGEAVHRKPSPGGYRA | 4 |
| VACCS-5p | GEAVHRKPSPGGYRAGDSLTLRAVWEGAGM | 5 |
| VACCS-6p | SRIPSKKSSLRKSTKSYKSSTSLKSLVPT | 6 |
| VACCS-11p | SPVASRGWVDTLKTPPSTTTGSPSWSPSWG | 11 |
| VACCS-13p | SSAVAVGGPTPRIGPTPSRSSSPTGRAWS | 13 |
| VACCS-14p | NLDLQHQKKRKKKKDKVETQNRSQRANSTE | 14 |
| VACCS-15p | KMGKRKSLKRRKLQELPKRRKLRKNSNLSH | 15 |
| VACCS-17p | NSPNGSRKRKKSSNFRINHGGLEPN | 17 |
| VACCS-20p | NLRRMVRGFIPSWIGTYIYLPKRKNLSYKR | 20 |
| VACCS-21p | TSRWTRKGTISLSPLLIMFIKFRKFRSPR | 21 |
| VACCS-23p | ATTPSTSLVRRKRWLFPQSRRQGAQNEGVL | 23 |
| VACCS-26p | MMRTKRLNKMASNQKIKLIRPRPKFRLASVDT | 26 |
| VACCS-28p | YLAYREEGEVPELAVHGLAGDAVPRPQTAG | 28 |
| VACCS-29p | SSRESWEPGGKQQWFHKVPGSELVAPSWRL | 29 |
| VACCS-30p | RDRDGDHGARQAPRVGSQHLSAGTEHYGRR | 30 |
| VACCS-32p | PAGDVAGVVVGHGVRHPHRPPGLPQVWKGK | 32 |
| VACCS-34p | PPGGRGGLPPSPLLRVLYETWEDASSSPGN | 33 |
| VACCS-37p | FIARTISGITSRPMTPTKRPFTVLSAVRIT | 34 |

### Example 2: Vaccine Efficacy Study

A randomized, blinded, placebo-controlled study with 2 treatment groups is performed. The data presented in this example demonstrate that the vaccines as described herein are unexpectedly superior in efficacy compared to traditional vaccines for canines.

The experimental unit is the individual dog. The primary efficacy endpoint is the cumulative incidence (CI) of dogs developing malignant neoplasia of any type at the end of the study period.

The secondary endpoints include: Incidence of adverse effects; Cumulative incidences of specific tumor types; Survival time following diagnosis of neoplasia, time from vaccination to diagnosis of a tumor, and tumor incidence after 2 years of the trial. Additionally, safety data, including the type and severity of adverse events, are collected.

Randomization: Eligible dogs are randomized in a 1:1 ratio to a vaccination or control arm following permuted block randomization methods. The randomization is stratified by study site and breed (spaniel vs. non-spaniel breed).

Blinding Procedure: Blinding is accomplished by using four treatment codes (two assigned to treatment, two assigned to placebo) provided by the Statistician to identify treatment groups. All personnel conducting observations, collecting data, and dispensing and administering treatment are blinded to treatment group.

The test article or placebo is provided in identical vials for administration at the study sites, and have identical volumes and appearances.

Blinding occurs at the time of dispensing the vaccine vial for administration.

SCHEDULE: Day 0 for each dog is defined as the day the Investigational Veterinary Product (IVP) or Control Product (CP) is first administered to the dog. Details of the schedule, the compositions, and subjects are set forth in Tables 3-6.

TABLE 3

Individual Dog Study Schedule

| | | | | | | |
|---|---|---|---|---|---|---|
| Time | History, PE | CBC, CC, Coagulogram, Urinalysis | PBMC | Serum | DNA Vaccine | Peptide Vaccine |
| Week 0 | X | X | X | X | X | |
| Week 2 | X | | | | X | |
| Week 4 | X | | | | | X |
| Week 6 | X | | X | X | | X |
| Month 6 | X | | X | X | | |
| Month 12 | X | X | | X | | X |
| Month 18, 30, 42, 54 | X | | | X | | |
| Month 24, 36, 48, 60 | X | X | | X | | X |

TABLE 4

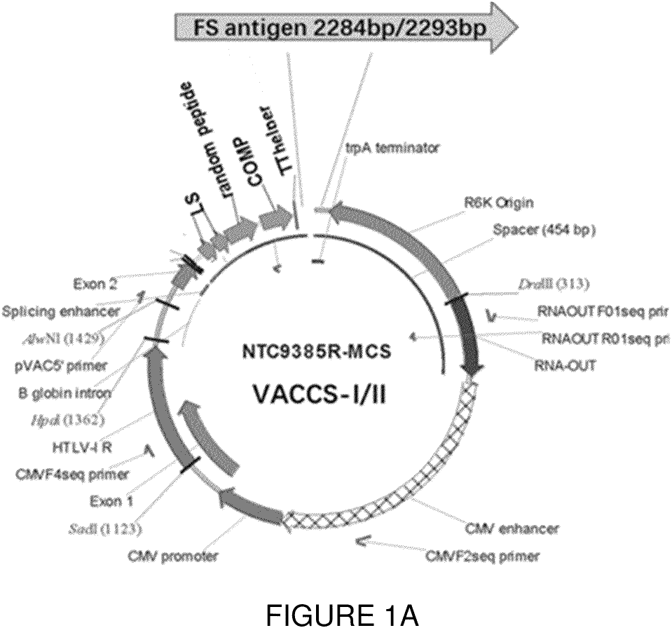

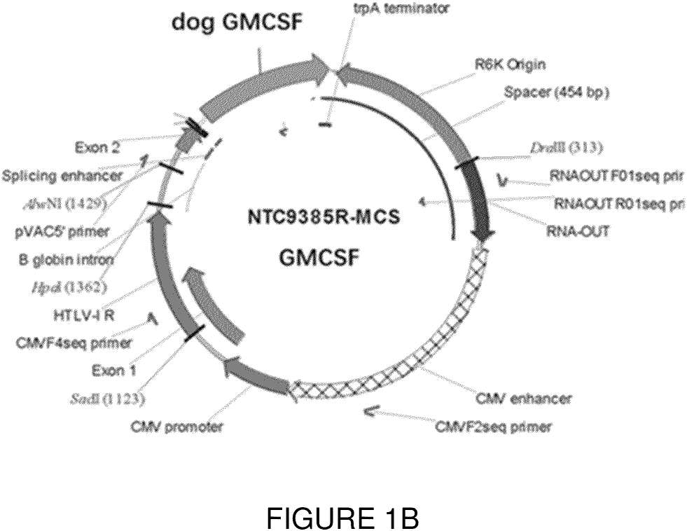

INVESTIGATIONAL VETERINARY PRODUCT

| | |
|---|---|
| DNA VACCINE | |
| Active pharmaceutical ingredient | VACCS-I plasmid +VACCS-II plasmid + GMCSF plasmid (See FIGS. 1A and 1B) |
| Dose form | Sterile solution in 200 µL PBS for injection (USP), using 100 µL for epidermal injection via PharmaJet Tropis ID |
| Strength | 4 mg/mL total DNA plasmid. 0.4 mg per dose. |
| Storage conditions: | Unopened vial: -20° C. away from light. |
| | After thawing: If the vaccine is not immediately used, it is stored at room temperature (20 to 25° C.) for up to 4 hours prior to use. If the storage period exceeds this, the drug solution is discarded and a new dose is prepared. |
| | The vial is a single use vial and does not contain a preservative. If there is remaining drug solution in the vial after withdrawing the required dose, the remaining material is discarded. |
| Active pharmaceutical ingredient | 20 peptide pool (See Example 1) + Hiltonol Adjuvant. |
| Dose form | Sterile solution in 800 µL PBS for injection (USP), for 500 µL intramuscular injection via PharmaJet Stratis IM. Sterile Hiltonol solution in 1 mL, for 200 µL intramuscular injection. |
| Strength | 800 µL of 0.5 mg/mL peptide pool per vial and 2 mg/mL Hiltonol per vial; 500 µL injection delivers 0.25 mg peptide pool; 200 µL injection delivers 0.4 mg Hiltonol/dose |
| Storage conditions: | Unopened peptide vaccine vial: -20° C. away from light. Unopened Hiltonol adjuvant vial: 4° C. away from light. After thawing: The vial is a single use vial and does not contain a preservative. If the vaccine is not immediately used, it is stored at room temperature (20 to 25° C.) for up to 4 hours prior to use. The Hiltonol vial is returned to 4° C. storage for up to 7 days prior to use. If the storage period exceeds these times, the vaccine and/or adjuvant is discarded and a new dose is prepared. |

TABLE 5

CONTROL PRODUCT

| | |
|---|---|
| DNA VACCINE | |
| Active pharmaceutical ingredient | None |
| Dose form | Sterile solution in 200 µL PBS for injection (USP), for 100 µL epidermal injection via PharmaJet Tropis ID |
| Strength | Not applicable |
| Storage conditions: | As per label. |
| PEPTIDE VACCINE | |
| Active pharmaceutical ingredient | None |
| Dose form | Sterile solution in 800 µL PBS for injection (USP), for 500 µL intramuscular injection via PharmaJet Stratis IM. Sterile Hiltonol solution in 1 mL, for 200 µL intramuscular injection. |
| Strength | 800 µL of 0.02 mg/mL negative control peptide per vial and 2 mg/mL Hiltonol per vial; 500 µL injection delivers 0.01 mg negative control peptide; 200 µL injection delivers 0.4 mg Hiltonol/dose |
| Storage conditions: | Unopened peptide vaccine vial: -20° C. away from light. Unopened Hiltonol adjuvant vial: 4° C. away from light. After thawing: The vial is a single use vial and does not contain a preservative. If the vaccine is not immediately used, it is stored at room temperature (20 to 25° C.) for up to 4 hours prior to use. The Hiltonol vial is returned to 4° C. storage for up to 7 days prior to use. If the storage period exceeds these times, the vaccine and/or adjuvant is discarded and a new dose is prepared. |

TABLE 6

STUDY ANIMALS

| | |
|---|---|
| Species: | Dog |
| Breed: | Select pure breeds (see below) and mixed breeds |
| Initial age: | Between 5 and 10.5 years old on Day 0 |
| Weight: | Dogs must weigh at least 5 kg |
| Sex: | Male or female, intact or neutered |
| Husbandry: | Normal infectious disease vaccination and general health care practices are permitted. |

### Inclusion and Exclusion Criteria

Inclusion: All of the following must be true for the dog to be included in the study: Dogs are between six and ten years old on Day 0; Dogs are mixed-breed dogs, or dogs belonging to one of the breeds listed below in Table 9; Dogs must weigh at least 5 kg on Day 0; No more than 2 dogs living in the same household may participate; Dogs must have received a wellness examination from a veterinarian within 12 months of Day 0, and at least 3 years of medical records from the regular veterinarian must be available for review; Adequate organ function as demonstrated by: Absolute neutrophil count (ANC) > 2,000 cells/µL, Hematocrit > 35%, Platelet count > 175,000 /µL, Normal serum creatinine, Normal bilirubin, Normal transaminases; Constitutional Clinical Signs General Performance score of 0 on Day 0 (VCOG-CTCAE v1.1); and Signed owner informed consent.

Exclusion: The presence of any one of the following would result in the dog being excluded from entry into the study: A history of any previous malignant neoplasm. A history of previous histologically and biologically benign neoplasia is not an exclusion criterion; Current evidence of likely, probable or definite malignant neoplasia on pre-enrollment screening; Concurrent co-morbidities, which in the opinion of the Investigator, has the potential to interfere with the ability to follow a patient for 5 years; Dogs that are receiving immunosuppressive therapy; Dogs that have been diagnosed with hyperadrenocorticism; Currently enrolled in another clinical trial; Owned by an Investigator or his/her staff or family; Constitutional Clinical Signs General Performance score ≥0 on Day 0 (VCOG-CTCAE v1.1); Pregnant, lactating or intended for breeding (male or female); Any other reason which according to the Investigator, would affect the safety of the dog or interfere with study procedures; Owner or family veterinarian unwilling or unable to furnish medical records for review; Owner unwilling to pursue necropsy evaluation if dog dies while still enrolled in study.

Post-Inclusion Removal (Withdrawal): Post-inclusion withdrawal may occur for the following reasons: Dog does not complete the initial series of 4 biweekly vaccinations, censored; Investigator or owner non-compliance with study procedures, censored; Unacceptable adverse event according to the opinion of the Investigator, censored; Owner elects to withdraw the dog from the study, censored; Investigator elects to withdraw the dog from the study, censored.

### Materials and Methods

Definition of Study Day: Day 0 for each dog is defined as the day on which the dog receives the first vaccine or placebo. All assessments and procedures subsequent to Day 0 are carried out within ± 5 days of the visit day specified for the first 8 weeks, and ± 2 weeks for the every-six-month rechecks thereafter.

Complete Blood Count: Complete blood counts (CBCs) are performed at the pre-enrollment screening visit and yearly thereafter. Whole blood is collected in EDTA anticoagulant tubes for routine hematology and analyzed. The sample is analyzed on the day of collection or, as soon as possible thereafter, in accordance with the laboratory’s usual practices. At a minimum, hemoglobin, PCV/HCT, red blood cell parameters, total and differential white cell counts and platelet counts are measured.

Clinical Chemistry: A clinical chemistry (CC) analysis is performed at the pre-enrollment screening visit and yearly thereafter. A blood sample is collected for clinical chemistry in a tube with no anticoagulant. Serum samples are stored in accordance with usual practices until analysis. Clinical chemistry tests are performed in accordance with the laboratory’s usual practices. At minimum, the clinical chemistry parameters measured include blood chemistry parameters including alanine aminotransferase, albumin, alkaline phosphatase, anion gap, aspartate aminotransferase, bilirubin (total), blood urea nitrogen, calcium (total), chloride, cholesterol, creatine kinase, creatinine, gamma glutamyl transferase, globulin, glucose, hemolysis index, lipemia index, phosphorus, potassium, sodium, and total protein.

Coagulogram: Whole blood is collected in sodium citrate anticoagulant tubes at the pre-enrollment screening visit and yearly thereafter. This is submitted for routine coagulation function analysis and analyzed. The sample is analyzed on the day of collection, in accordance with the laboratory’s usual practices. Activated partial thromboplastin time and prothrombin time are measured.

Urinalysis: Urinalysis is performed at the pre-enrollment screening visit and yearly thereafter. Urine is collected by free catch, catheterization or cystocentesis and refrigerated until submitted for analysis. The sample is analyzed. At a minimum, as specific gravity, protein, blood, bilirubin, glucose, and presence of WBC, RBC, casts and epithelial cells are tested.

Peripheral Blood Mononuclear Cells: A blood sample of at least 20 mL is collected into sodium heparin tubes pre-treatment, at 6 weeks, and at the 6 month recheck. PBMC is isolated and stored.

Serum: Approximately 2 mL serum (4-6 mL whole blood) is collected in a tube with no anticoagulant, processed and banked every six months.

Radiography: Thoracic (3 views) radiographs are obtained at the pre-enrollment screening visit. Radiographs are evaluated.

Abdominal Ultrasound: Full abdominal ultrasound is obtained at the pre-enrollment screening visit. Ultrasounds are evaluated.

Physical Examination: A physical examination, to include assessment of the vaccination site if appropriate, occurs at the pre-enrollment screening visit and as part of each subsequent visit. Abnormalities are recorded.

Tumor Tissue: In patients developing confirmed neoplasia, samples are collected as soon as possible postoperatively to preserve RNA integrity. Three samples are obtained from the tumor in such a way that surgical margins are not disturbed and snap-frozen in liquid nitrogen in individual cryovials. Whenever possible, 1-2 normal tissue samples are obtained and snap-frozen in liquid nitrogen. Tissues are placed in formalin for later centralized pathology review.

Test Articles and Dosage: A plasmid DNA vaccine, either encoding the frameshift antigens of interest or a control insert is administered intradermally via a PharmajetTM needle-free injection device (Tropis) at Weeks 0 and 2. 300 µg of plasmid DNA and 200 µg canine GM-CSF plasmid in PBS are administered per injection in a volume of 100 µL. A peptide pool vaccine or scrambled peptide control vaccine (225 µg peptide pool vaccine or 10 µg placebo peptide in 500 µL) is administered intramuscularly via a Pharmajet™ needle-free injection device Weeks 4 and 6, and yearly thereafter (see Example 1 for amino acid sequence of frameshift peptides) along with 400 µg (200 µL) Hiltonol adjuvant injected intramuscularly using a 21 gauge, 1″ needle and syringe immediately before peptide vaccine/placebo administration.

### Administration Procedure

DNA Vaccine / Placebo: The vial of test article or placebo is removed from the freezer and brought to room temperature. 100 µL of the solution is administered intradermally via a Pharmajet Tropis™ needle-free injection device in the sparsely-haired skin overlying the right (or left) medial thigh. If needed, fur is clipped where the vaccine is administered to ensure contact with the injection device.

Peptide Vaccine / Placebo: The vials of test article or placebo are removed from the freezer and brought to room temperature. The Hiltonol adjuvant is brought to room temperature and 200 µL is administered intramuscularly in the left (or right) cranial thigh (quadriceps muscle) using a 21 gauge, 1″ needle and syringe immediately before peptide vaccination. The peptide pool solution contains 0.225 mg of peptide pool vaccine or 0.01 mg control peptide vaccine per dose. 500 µL of this solution is administered intramuscularly via a Pharmajet Stratis™ needle-free injection device in the same site as the Hiltonol injection. If needed, fur is clipped where the vaccine is administered to ensure contact with the injection device.

Dose Limiting Toxicity: A DLT is defined as any non-hematologic Grade 3 or higher toxicity. The exceptions that are not dose limiting are: Abnormalities clearly not related to study drug, such as physical injury, congenital abnormality; Anorexia, vomiting or diarrhea remediable within 24 hours by medical therapy; Injection site toxicities that are significant and/or extensive but that may not meet Grade 3 criteria; A hematologic DLT is defined as an uncomplicated (e.g., no sepsis or bleeding) Grade 4 neutropenia or thrombocytopenia. If sepsis, bleeding or another complication occurs, the toxicity is graded according to the VCOG-CTCAE v1.1 and is considered DLT if Grade 3 or 4.

Dose Modification or Delay: A dose delay of more than 5 days (during the first 6 weeks of the study) or more than 2 weeks (during the remainder of the study) is considered a protocol deviation.

Concomitant Medications: From Day 0 to the last day of the study, the owner is requested to notify the Investigator within 7 days of any visit for veterinary care for the diagnosis or treatment of any medical, surgical or traumatic condition, and medical records from this visit are obtained. Concomitant treatments approved by the Investigator are permitted for the supportive care of the patient during the study. For any life-threatening adverse events, appropriate supportive care is immediately instituted. All concomitant treatments are recorded.

Not allowed: Antineoplastic chemotherapy; Antineoplastic immunotherapy or radiation therapy; Investigational medications.

Allowed: Topical corticosteroids for treatment of ear or skin disease; Medical treatment as deemed appropriate and necessary to treat adverse events or unrelated conditions; Antibiotics to treat specific infections; Sedation/anesthesia as needed for any procedures required during the study; Heartworm/flea/tick preventative, dewormers, routine vaccinations; Medications for allergies and atopic dermatitis, including Apoquel (oclacitinib) and Cytopoint but not including chronic corticosteroids or cyclosporine; Over-the-counter supplements.

### Methods and Observations

Pre-enrollment Screening Visit, Day -28 to Day 0: Dogs fulfilling the inclusion criteria undergo laboratory tests (hematology (CBC), coagulogram, CC and urinalysis) to ensure the other inclusion criteria are met.

Animal Details and Animal History including details of previous medical conditions are recorded. A Physical Examination is performed and body weight is recorded. 3-view thoracic radiography and abdominal ultrasound is performed to ensure the absence of occult neoplasia or other disease impacting study eligibility. The results of these examinations are recorded.

If there are lesions noted on thoracic radiography and/or abdominal ultrasound where neoplasia is possible and cannot be completely ruled out, dogs are rescreened in 4 to 6 weeks at cost to the owner to determine progression or resolution of these lesions.

Study period: Week 0 Enrollment Visit: The Owner must have provided written informed consent in order to enroll in the study.

Inclusion and exclusion criteria are assessed. Only animals that fulfill all of the inclusion criteria and to which none of the exclusion criteria apply are enrolled in the study.

Concomitant treatments are recorded.

An Owner History and Physical Examination are performed and body weight recorded. Medical records from the family veterinarian are reviewed and any historical medical/surgical conditions are documented.

2 mL serum is collected and stored.

At least 20 mL heparinized anticoagulated blood is submitted for PBMC isolation.

Plasmid DNA vaccine or placebo is administered intradermally in the right medial thigh.

Week 2 visit: A Physical Examination is performed and body weight recorded. A detailed Owner History is collected and any reported adverse events recorded and prospectively graded according to the VCOG-CTCAE v1.1.

Plasmid DNA vaccine or placebo is administered intradermally in the left medial thigh.

Week 4 visit: A Physical Examination is performed and body weight recorded. A detailed Owner History is collected and any reported adverse events recorded and prospectively graded according to the VCOG-CTCAE v1.1. Hiltonol is administered IM using a 21 gauge, 1″ needle and syringe into the right lateral cranial thigh (quadriceps muscle). Immediately after, peptide vaccine or placebo is administered IM (PharmaJet Stratis IM) at the same site.

Week 6 visit: A Physical Examination is performed and body weight recorded. A detailed Owner History is collected and any reported adverse events recorded and prospectively graded according to the VCOG-CTCAE v1.1.

At least 20 mL heparinized blood is submitted for PBMC isolation.

2 mL serum is collected and stored.

Hiltonol is administered IM using a 21 gauge, 1″ needle and syringe into the left lateral cranial thigh (quadriceps muscle). Immediately after, peptide vaccine or placebo is administered IM (PharmaJet Stratis IM) at the same site.

Month 6 visit: A Physical Examination is performed and body weight recorded. A detailed Owner History is collected and any reported adverse events recorded and prospectively graded according to the VCOG-CTCAE v1.1.

At least 20 mL heparinized blood is submitted for PBMC isolation.

2 mL serum is collected and stored.

Month 12 visit: A Physical Examination is performed and body weight recorded. A detailed Owner History is collected and any reported adverse events recorded and prospectively graded according to the VCOG-CTCAE v1.1.

Blood and urine are collected for CBC, coagulogram, clinical chemistry and urinalysis.

2 mL serum is collected and stored.

Hiltonol is administered IM using a 21 gauge, 1″ needle and syringe into the left lateral cranial thigh (quadriceps muscle). Immediately after, peptide vaccine or placebo is administered IM (PharmaJet Stratis IM) at the same site.

Subsequent visits: Subsequent visits are performed on an every-six-months basis, as long as the dog has not developed confirmed neoplasia and the dog has not experienced any dose limiting toxicities.

Procedures performed at these visits are identical to those described in the Month 6 and Month 12 visits above. The only exception is that blood is not collected for PBMC isolation at these visits.

Follow-Up and Development of Neoplasia: Dogs undergo thorough physical examination every 6 months for the duration of the study. Owners are advised to seek visit with their family veterinarian as soon as possible if any new masses or lymphadenopathy are discovered in between scheduled visits. Any peripherally accessible masses or enlarged lymph nodes are investigated according to standard oncologic practice. Whenever possible, tumor tissue is collected from all cytologically confirmed or suspicious, or histologically confirmed neoplasms, and processed. Dogs undergoing tumor surgery have tumor tissue collected as soon as possible to preserve RNA integrity. Tumors are submitted via the anatomic pathology services at the respective VTHs. A request is made for two extra H&E stained slides to be prepared and sent to CSU for centralized review.

Owner Observations: Owners are provided with unique access to the EDC and are asked to record animal observations one week following each vaccination. These observations include appetite and attitude, episodes of vomiting, diarrhea, excessive urination, excessive drinking or any other observation.

Owners are requested to notify the Study Site immediately if their dog presents to any veterinarian for diagnostics or treatment of any medical, surgical or traumatic condition. The RDVMs of record are contacted twice yearly to determine if any visits have taken place, and the diagnostics, procedures and therapies instituted are recorded. This will include dispensing of routine preventative medications (e.g., parasite prevention) and routine infectious disease vaccinations.

Study Withdrawal: The primary reason for withdrawing from study is noted.

If withdrawal occurs prior to Month 12, the Investigator conducts all the observations and procedures that have been listed for the Month 12 visit, if possible.

Adverse Events, if the reason for withdrawal, are recorded.

Adverse Events: An adverse event (AE) is any observation in animals that is unfavorable and unintended and occurs after the use of a veterinary product or investigational veterinary product, whether or not considered to be product related.

Documentation of Adverse Events: The adverse event reporting period is from the first treatment until the dog is withdrawn or leaves the study.

All AEs are recorded including onset and stop dates, interventions, medication required and grading of the severity using VCOG-CTCAE v1.1 as well as an adaptation of the FDA Guidance for Industry: Toxicity Grading Scale for Healthy Adult and Adolescent Volunteers Enrolled in Preventative Vaccine Clinical Trials (9/2007). Any dog with an on-going adverse event is monitored until the event has resolved, has become chronic and/or stable, whether the dog has been withdrawn from the study or not.

Evaluation of Causality: Causality of all AEs in relation to the IVP are assessed by the investigator using the classifications probable, possible, unlikely, unknown, no assessment.

Serious Adverse Events: A serious adverse event (SAE) is an adverse event that is fatal, or life-threatening, or requires professional intervention, or causes prolonged or permanent disability, or disfigurement.

Notification of Serious Adverse Events: Within 24 hours of identification, the Investigator reports to the Sponsor Monitor all SAEs. This responsibility for notification is valid until a dog leaves the study.

The Investigator completes and submits to the Sponsor Monitor an SAE report for all SAEs regardless of causal relationship to the IVP. The initial report is completed by a follow-up report as soon as the Investigator obtains additional information about the event. Any serious adverse event that is still on-going at study end continues to be followed even after the dog’s participation in the study is over. SAEs are followed until they resolve or until the Investigator assesses them as chronic and/or stable.

Death or euthanasia: If an animal dies or requires euthanasia prior to study end, the owner’s consent to perform a necropsy of the dog is requested. The reason for euthanasia or the cause of death is described by the Investigator. If a necropsy is allowed, gross and histopathologic examination is performed by a pathologist and the report provided to the Investigator.

Fate of animals at study end: Dogs remain in the custody of their owners on completion of the study.

Statistical considerations: This is a randomized, multi-institutional, controlled double-blind trial with the primary objective to evaluate the efficacy and safety of a novel frameshift peptide-derived vaccine, for the prevention of tumor development in dogs. The primary efficacy endpoint is the cumulative incidence (CI) of dogs developing malignant neoplasia of any type at the end of the study period. Secondary endpoints include the incidence of adverse effects, cumulative incidences of specific tumor types, and overall survival following diagnosis of neoplasia. The groups are designated Placebo (N=400) and Vaccination (N=400). A formal statistical analysis plan (SAP) is developed before the database lock. It includes detailed descriptions of summaries and mock-ups of tables, listings, and figures to be included in the clinical study report.

Analysis Populations: Analyses are conducted in the Intent-To-Treat (ITT), Per Protocol (PP) and Safety populations: The ITT population includes all randomized dogs. The ITT population serves as the primary analysis population for the analyses of all primary and secondary efficacy endpoints of this trial. The PP population includes all dogs of the ITT population who meet adequate study treatment compliance, defined as the completion of at least the initial series of 4 biweekly vaccinations, and have no major protocol violations. The PP population serves as the secondary analysis population for all primary and secondary efficacy endpoints of this trial. The Safety population consists of all dogs who receive at least one dose of vaccination.

Sample Size and Power Calculation: The primary hypothesis of this randomized, controlled double-blind trial is that cancer vaccination decreases the CI for developing malignant neoplasia of any type at the end of the study period. Specifically, it is anticipated that vaccine results in a relative reduction of at least 30% in the CI for the development of malignant neoplasia of anytime when compared to the control arm.

A time to event analysis accounting for competing risks is conducted to compare the CIs for the development of malignant neoplasia of any type between the vaccine treatment and the control arm. In this analysis, a clinical diagnosis of malignant neoplasia of any type or clinically confirmed death event due to malignant neoplasia of any time, is defined the event of interest. Death events, due to any other causes or study discontinuation are defined as competing risks. Approximately 25% of the dogs in the control group have an event of interest by the end of the 5-year study period. The CI for competing risks is assumed to be between 50-60%.

An interim analysis for superiority is conducted at the end of years 2, 3 and 4 using a Hwang-Shih-DeCani gamma spending function. A total sample size of 800 eligible dogs is proposed for this trial. This sample size is adequate for detecting the anticipated decrease of at least 30% in the CI for the development of malignant neoplasia of any type in the vaccination arm when compared to the control arm at the two-sided 0.05 significance level. The following table shows the attainable power level for rejecting the null hypothesis that the rate ratio of the CI is one using a stratified log-rank test accounting for competing risks under the following assumptions: (1) a two-sided 0.05 significance level, (2) a sample size of 400 eligible dogs per study arm, (3) an attrition rate/unevaluable rate of 10%, (4) an anticipated CI for the development of malignant neoplasia of any type of 25% in the control arm at the end of the follow-up period, (5) 30-50% decrease in the CI for the development of malignant neoplasia of any type in the vaccination arm when compared to the control arm, (6) CIs for competing risks in the control arm ranging between 50-60% both the control and vaccination arm, (7) a total study period of 5 years including a 2 year accrual period, (8) uniform accrual pattern over 2 years, (9) time to diagnosis of malignant neoplasia and time to competing risks follow an exponential distribution, and (10) an interim analysis for superiority at the end of year 2, 3 and 4 using a Hwang-Shih-DeCani spending function (3 interim analyses plus 1 final analysis) with γ=-4.

TABLE 7

Attainable power level for detecting a decrease of at least 30% in the CI for the development of malignant neoplasia of any type at the two-sided 0.05 significance level under various scenarios with a total sample size of 800 eligible dogs
Relative reduction in CI for the development of malignant neoplasia of any type in vaccination arm compared to control arm

| | | | | | |
|---|---|---|---|---|---|
| CI for competing risks | 30% | 35% | 40% | 45% | 50% |
| 50% | 77% | 88% | 95% | 98% | >99% |
| 55% | 80% | 91% | 96% | 99% | >99% |
| 60% | 84% | 93% | 98% | 99% | >99% |

Hence, with the proposed sample size of eligible 800 dogs, the anticipated decrease of at least 30% in the CI for the development of malignant neoplasia of any type in the vaccine treatment arm is detected with 80-99% power if the CI for the competing risks is at least 55%. If the CI for the competing risks is between 50-55%, then the anticipated reduction of 30% in the CI for the development of malignant neoplasia of any type is detected with at least 77% power at the two-sided 0.05 significance level. These calculations are based on the log-rank test accounting for competing risks, assuming that the time to the diagnosis of malignant neoplasia of anytime and time to competing risk failures are exponentially distributed.

Accrual and Recruitment: accrual of 800 eligible dogs is completed within 2 years. The total study period is 5 years, there is a minimum follow-up period of 3 years.

Analysis Plan: Demographics and Baseline Characteristics: Demographic variables and baseline characteristics measured on a continuous scale are summarized in terms of number of subjects, means, standard deviations, medians and ranges, stratified by study arm. A two-sample t-test or nonparametric Wilcoxon rank sum test is used to conduct the comparisons between arms. Categorical demographic variables and baseline characteristics are summarized in tabular format and compared between arms using a chi-square of Fisher’s exact test. Significant imbalances found between study arms are considered as covariates in exploratory analyses of primary and secondary efficacy endpoints.

Analysis of Primary Endpoint: The CI for the development of malignant neoplasia of any type at the end of follow-up period is calculated for each arm using the Kalbfleisch and Prentice method and reported along with the corresponding 95% confidence interval. In this analysis, a clinical diagnosis of malignant neoplasia of any type or a confirmed death due to malignant neoplasia of any time, is defined the event of interest. Death events, due to any other causes (non-cancer) or study discontinuations is defined as competing risks. The primary analysis is the comparison of the CI for the development of malignant neoplasia of any type between the vaccine treatment and the control group. This comparison is conducted using the proportional hazard model for competing risk data. The randomization strata, study site, and breed is included as covariates in the model. The primary analysis is conducted in the ITT analysis population.

In addition, the following sensitivity analyses are performed to explore the robustness of the primary confirmatory analysis: (1) Conducting the analyses described above in the PP analysis population. (2) Conducting a proportional subdistribution hazard analysis for competing risk data analysis where baseline characteristics are included as covariates. The selection of baseline characteristics to be included in the multivariate analysis are determined before finalizing the SAP.

Analysis of Secondary Endpoints: The CI for the development of specific tumor types is analyzed using the proportional subdistribution hazard model for competing risks in the same way as described for the primary efficacy analysis. Overall survival following a diagnosis of neoplasia is estimated using the Kaplan-Meier method and compared between study arms using the stratified log-rank test and multivariate Cox proportional hazard regression analysis where baseline characteristics (including age and breed) are included as covariates. The selection of covariates is specified in the final SAP. The confidence intervals for the median survival are calculated using the Brookmeyer-Crowley method.

Safety Endpoints: Safety endpoints are summarized in tabular format, stratified by type. Chi-square of Fisher’s exact test is used to compare adverse event rates between study arms. Longitudinal changes are evaluated using a generalized linear mixed effects model with participant specific random effects. Safety outcomes measured on a continuous scale are summarized in terms of means, standard deviations, medians and ranges and compared between study arms using a two-sample t-test or nonparametric Wilcoxon rank sum test.

Interim Analysis: An interim analysis for the primary efficacy endpoint is conducted for evaluating superiority at the end of years 2, 3 and 4 using Hwang-Shih-DeCani spending function with γ=-4, wherein there are up to 3 interim analyses and one final analysis. The interim analyses are conducted in a blinded fashion and reviewed by the independent DSMB.

TABLE 8

Stopping boundaries for superiority using a Hwang-Shih-DeCani spending function with γ=-4
Year

| | 2 (Interim Analysis 1) | 3 (Interim Analysis 2) | 4 (Interim Analysis 3) | 5 (Final Analysis) |
|---|---|---|---|---|
| Nominal alpha | 0.0037 | 0.0075 | 0.0183 | 0.0444 |

The DSMB reviews the interim efficacy results and provides the recommendation on whether the study should be unblended and/or discontinued early. Operational details for the DSMB are detailed in the DSMB Charter.

Post-hoc Monitoring: The status of dogs in the trial after it terminates continues to be monitored. The database information is used to send regular inquiries to owners and participating vets. Information on tumor incidence, type of tumor, survival and other health incidences is polled. The objective is to determine if there are other significant positive or negative effects on the participating dogs.

### Safety Evaluation

Analysis of Adverse Events: An overview of all AEs including severity, relationship to study drug, serious AEs and AEs leading to deaths or withdrawals is presented. All AEs are listed in animal data listings by date.

Analysis of Laboratory Safety Data: Laboratory safety (hematology and clinical chemistry) data are graded according to VCOG-CTCAE v1.1 and listed according to VCOG-CTCAE grade. All clinically significant deviations from normal ranges are reported as adverse events as stated above.

### Protocol Amendments and Deviations

Amendments: Protocol amendments are defined as a written change to the protocol prior to implementation. They are described and justified, and signed by the Sponsor, the Statistician (if relevant to statistics) and the Investigator. Signatures received via facsimile or electronically are acceptable. An explanation of each amendment is included in the FSR.

Deviations: Protocol deviations are defined as unintended departures from the protocol. If a deviation occurs, it is described, the reason for the occurrence and the potential impact upon the study are documented and signed and dated by the Investigator. The Investigator notifies the Sponsor within 24 hours of the identification of the deviation. An explanation of each deviation is included in the FSR.

TABLE 9

List of pure breed dog breeds eligible for study inclusion
Afghan hound
Airedale terrier
Alaskan malamute
Basset hound
Beagle
Bernese mountain dog
Borzoi
Boston Terrier
Boxer
Briard
Bullmastiff
Cocker spaniel
Corgi
Deerhound
English setter
Field spaniel
Flat-coated retriever
French bulldog
German shepherd
German shorthaired pointer
Giant schnauzer
Golden retriever
Gordon setter
Great Pyrenees
Irish setter
Irish water spaniel
Irish wolfhound
Italian spinone
Keeshond
Labrador retriever
Leonberger
Newfoundland
Norwegian elkhound
Nova Scotia duck tolling retriever
Old English sheepdog
Petit bassett griffon Vendeen
Rhodesian ridgeback
Rottweiler
Saluki
Scottish terrier
Shetland sheepdog
Siberian husky
Springer spaniel
Staffordshire bull terrier
Standard poodle
Tibetan terrier
Viszla
Welsh terrier
West highland white terrier

Description of Peptide Boost Vaccine: Frameshift vaccine peptides are received and a pool of peptides are prepared in which the peptides are suspended into a single pool in 1x PBS for injection. The peptide pool is produced under aseptic conditions in a biosafety cabinet and transferred to a sterile flask. The solution is sterile filtered (0.2 micron) prior to dispensing into sterile 2 mL vials, which also takes place under aseptic conditions. A portion of the final vialed vaccines are shipped to Pace Analytical for endotoxin testing, which meets the specifications of USP <85>, and sterility testing, which meets the specifications of USP <71>. Upon receipt of this information, the product is released to generate a CoA that contains the information listed below, and store the product at -80° C. The boost vaccine and placebo vaccine are shipped to clinical sites on dry ice. The adjuvant (PolyIC:LC, tradename Hiltonol) was produced by Oncovir under cGMP production and shipped to ASU. Upon release of the peptide boost vaccine, the Hiltonol adjuvant is shipped to clinical sites on cool packs.

TABLE 10:

| | | |
|---|---|---|
| QC step | Methods | Specification |
| Appearance | Visual inspection | Clear, no visible particles |
| Concentration A280 | UV absorption | 0.4 - 0.6 |
| Endotoxin assay | USP <85> | < 50 EU/mL |
| Sterility Test | USP <71> | No visible growth |

### SOP for Serum Collection, Separation and Storage

Materials and equipment used in the studies include: 5-mL red top tube (BD Vacutainer cat # 366430 or similar); 2-mL cryovial (Sigma cat #CLS430488 or similar); ThermoFisher Life Technologies Ultrapure Glycerol (catalog# 15514-011); Sodium Azide (Sigma CAS# 26628-22-8); Prepare sterile glycerol with 0.02% sodium azide.

Autoclave an appropriate amount of glycerol (not to exceed 20 minutes at 121° C.)

A 0.2% solution of sodium azide in DI sterile water is prepared.

An appropriate amount of 0.2% sodium azide solution is added to the autoclaved glycerol to obtain a 0.02% final concentration of sodium azide solution in glycerol, and stored at room temperature.

To perform the study, blood is drawn into a red top tube and inverted 5-10 times. The blood sample in the red top tube is incubated for 30 minutes at room temperature to allow the blood to clot. The tube is then centrifuged for 10 minutes at 1,000-2,000 x g. The top serum layer is removed in Vacutainer tube, and about 0.5 mL is placed into a labeled 1.8-mL cryovial. About 0.5 mL of prepared glycerol/0.02% sodium azide solution is added to the serum samples in the 1.8-mL cryovial and mixed. Each serum sample is diluted to about 50% serum and 50% prepared glycerol/0.02% sodium azide solution to a 50/50 v/v. The diluted samples are retained at -20° C.

### SOP for PBMC Collection, Processing and Storage

Materials and equipment used in this study includes: 10 mL sodium heparin (green top) tubes (BD Vacutainer cat # 366480 or similar); 2-mL cryovial (Sigma cat #CLS430488 or similar); Lymphocyte Separation Medium (LSM) (Mediatech 25-072-CV); Sterile Phosphate-buffered saline (multiple vendors or lab-made); EDTA (multiple vendors); Trypan Blue (multiple vendors); ACK lysis buffer (ThermoFisher Cat# A1049201); DMSO (multiple vendors); Heat-inactivated fetal bovine serum (multiple vendors); Mr. Frosty (ThermoFisher Cat# 5100-0001).

To perform the study, about 20 mL heparinized whole blood is obtained prior to sedation/anesthesia. Under the laminar flow hood, the heparinized sample is placed in a 50 mL polypropylene conical centrifuge tube. The sample in each tube is brought to two times its volume with room temperature PBS/5 mM EDTA and mixed gently. The blood is underplayed with 8 mL LSM by placing the pipette all the way to the bottom of the tube and very slowly ejecting it beneath the blood/PBS mixture. The blood and LSM are not allowed to mix together. The quality of separation is dependent on a sharp interface between the diluted blood and density gradient.

The tube is centrifuged at 400 × g (1600 RPM - Allegra 6R) at room temperature for 15 minutes with no brake. Centrifugation sediments erythrocytes and polymorphonuclear leukocytes and the mononuclear lymphocytes are retained above the LSM. Using a pipet, the top layer of clear plasma is aspirated to within 2-3 mm above the lymphocyte layer. Using a pipet, the lymphocyte layer plus about half of the LSM layer below it are aspirated, and transferred it to a new centrifuge tube. A 5x volume of PBS/EDTA solution is added to the lymphocyte layer in the centrifuge tube and centrifuged for 5 minutes at room temperature (18-25° C.) at a speed sufficient to sediment the cells without damage (160-260 x g (1200-1400 rpm)). Washing the cells removes LSM and reduces the percentage of platelets. The supernatant is poured off. If pellet is blood tinged, small volumes (1.5 mL - 3 mL) ACK lysis buffer are added, and vortexed until cells are suspended in the solution, and incubated at room temperature or at 37° C. incubator/water bath for 5-8 minutes. The cells are vortexed for a few seconds, then PBS/EDTA is added to fill the tube. The tube is then centrifuged for 5 minutes at room temperature (18- 25° C.) at (1400 rpm). The supernatant is poured off into a beaker containing bleach. The cells are washed once with PBS/EDTA. An aliquot is counted using Trypan Blue, and the pellet is resuspended in FBS at a concentration of 2 × 10⁷ cells/mL.

A 2X freezing medium is prepared by adding DMSO to FBS at a ratio of (16% DMSO and 84% FBS) (The 2X freezing medium is diluted to 1X in the cryovial once added to the cells; therefore, the final concentration in the cryovial with cells is 8% DMSO in 92% FBS). 1 mL of 2X freezing medium include 840 µL FBS + 160 µL DMSO. 500 µL of cell/FBS mixture is added into a cryovial. 500 µL of the 2X freezing media is slowly added to the cryovial, the tube is capped and inverted gently to mix. Immediately after the freezing media is added to the cells in the cryovial, the cryovial is placed into a -80° C. freezer. The cells remain in the freezer for at least 24 hours before placing into a liquid nitrogen Dewar.

For SOP for collection, processing and storage of tumor tissue, the following materials and equipment are used: 2-mL cryovials (Sigma cat #CLS430488 or similar); 30-mL prefilled formalin jar (Leica cat # 3800770 or similar); Tissue cassettes (Leica Cat# 3802631 or similar); Liquid nitrogen and appropriate container; 100-mm Petri dishes (multiple sources); Sterile forceps, scissors and/or #10 scalpel blade.

For precollection set up, 3 cryovials labeled for frozen tumor, 1 cryovials labeled for frozen normal, 1 jar of formalin and the 3 tissue cassettes Sterile gloves, 2 Petri dishes (to separate stroma and tumor tissue) 2 #10 blade (one for stroma, one for tumor), and an Instrument pack containing 2 forceps are used. A blade handle or Michele Trephine may also be used as necessary. Liquid nitrogen is placed in a large Styrofoam container and some in a cryo cup. Cryovials are labeled with VACCS case number, either “tumor” or “normal”, and dated. Stickers are placed on a formalin jar with VACCS case number and date. Three tissue cassettes are obtained and labeled in pencil with the VACCS case number and either “tumor” or “normal”.

### Collection of Tissue Samples

Prior to surgery, OR nurses are informed of the intent to collect samples by flagging the case with the Tissue Archiving task option in the Surgery Worklist or by verbal confirmation. The OR nurses alert via PTT when the tumor is off and ready for collection.

Labeled cryovials, formalin jar, and tissue cassettes are placed in the hood.

Sterile gloves, Petri dishes, blades, and instrument pack are taken to the OR nurses’ station. A blade is used to cut enough tissue without compromising surgical margins, and is kept separate from normal tissue in another Petri dish.

Normal tissue (performed first to avoid tumor contamination): two pieces are cut to the size of a pencil eraser, placed in a single cryovial and placed in liquid nitrogen. While they freeze, one more piece is cut (thickness of a nickel) and placed in the tissue cassette.

Tumor tissue: Within 15 minutes of collection, using a new blade, three pieces are cut the size of a pencil eraser, placed into individual cryovials and placed in liquid nitrogen. While they freeze, two pieces are cut (thickness of a nickel) and placed one in each tumor tissue cassette. The cassette is closed and placed in formalin, and items are then removed from the hood.

The frozen samples all go into the correct box, and placed at -80° C. A paper box map with location of samples is updated.

The formalin sample is stored at 4° C. or room temperature.

Any remaining tumor samples are used for routine histopathology, special collection requests or put in the biohazard bag and autoclaved before disposal. Large body parts such as limbs should be returned to the OR nurses’ station for regular pathology submission.

### Example 3: Safety Determination

At year 2 of the trial, a safety of the vaccine was evaluated by an independent Data Safety Monitoring Board. Safety findings are presented in this example. In summary, it was importantly discovered that there was no concern for vaccine-associated adverse events. One of the concerns for a preventative vaccine with FSPs was if the FSPs happened to be self-antigens, they may cause autoimmune disease. FSPs generated by RNA mis-processing are not natural peptides, but neoantigens foreign to the immune system. The finding that no evidence of such vaccine events occurred in about 350 dogs receiving the vaccine corroborates this claim.

Vaccination Against Canine Cancer, Data and Adverse Event Data Summary: This example includes data acquired from dogs enrolled during an approximately two-year period, and represents data extracted from the physical exam, laboratory evaluation, and any health concerns requiring a veterinary visit.

Enrollment Data: Total consented = 672 dogs. Total dogs included in this example = 537. Dogs not completing initial vaccinations = 135 (20%). Non-completion of initial vaccination was due to various reasons, such as due to tumor identification at screening, poor temperament, owner compliance, or relocations. Data presented in this example were drawn from all dogs completing initial vaccination series.

TABLE 11

Summary Demographics

| | | | | |
|---|---|---|---|---|
| Weight | Mean = 27.7 +/-11.5 Kg | Median = 27.7 Kg | Range: 5.9-49.8 Kg | |
| Age at Consent | Mean = 7.6 +/-11.4 yrs | Median = 7.4 yrs | Range: 5.5-11.5 yrs | |
| Sex | F-12 | F/S - 273 | M-34 | M/C -218 |
| Breeds | Mix 216 | GR 83 | LbR 63, | GSD 14 |
| | BMD 12 | GshP 11 | Spdl 10 | Viz 9 |

TABLE 12

Cancer Diagnoses

| | |
|---|---|
| Benign | 20 |
| Malignant | 48 |
| Mast cell tumors (MCT) | 11 |
| Soft-tissue carcinoma (STS) | 7 |
| Hemangiosarcoma (HSA) | 5 |
| Feline lymphoma (LSA) | 3 |
| Other | 22 |

TABLE 13

Deaths

| | |
|---|---|
| Cause of Death: Cancer | 8 (HSA 3, Hep Ca 2, Histio Sarc 2, Chemode 1) |
| Cause of Death: Other | 14 |
| Total | 22 (16 necropsies) |

TABLE 14

Adverse Events (AE) Summary:

| | |
|---|---|
| Total AE | 986 |
| Unlikely, Unclear, Unknown relationship to vaccine administration | 560 |
| Somewhat or likely related to vaccine administration | 190 |
| Unrelated | 236 |

TABLE 15

Adverse Events by Grade

| | |
|---|---|
| .Grade 4 | 0 |
| Grade 3 | 12 (5 were unlikely, 2 unclear/unknown; 5 were somewhat/ likely) |
| Grade 2 | 100 (86 were unlikely, unknown; 14 were somewhat/ likely) |
| Grade 1 | 872 (699 were unlikely, unknown; 173 were somewhat/likely) |

TABLE 16

Adverse Events as a function of Grade and Vaccine Type

| Vaccine | Count of Grade |
|---|---|
| A | 216 |
| 1 | 189 |
| 2 | 26 |
| 3 | 1 |
| B | 233 |
| 1 | 204 |
| 2 | 26 |
| 3 | 3 |
| C | 279 |
| 1 | 247 |
| 2 | 27 |
| 3 | 5 |
| D | 258 |
| 1 | 234 |
| 2 | 21 |
| 3 | 3 |
| Grand Total | 986 |

TABLE 17

All Grade 3 Adverse Events

| VACCS ID | Type of Adverse Event | Attribution of Adverse Event |
|---|---|---|
| 400001 | Fever | Very likely vaccine related |
| 400002 | Fever | Very likely vaccine related |
| 400001 | Thrombocytopenia | Somewhat likely vaccine related |
| 40258 | Lethargy/fatigue/general performance | Unclear reaction to vaccine |
| 60011 | Neuropathy: cranial nerve | Unclear reaction to vaccine |
| 40142 | Dermatitis | Unclear reaction to vaccine |
| 40033 | Alkaline phosphatase | Somewhat unlikely vaccine related |
| 40099 | Seizure | Very unlikely vaccine related |
| 20067 | Pain | Very unlikely vaccine related |
| 20057 | ALT | Very unlikely vaccine related |
| 600005 | Diagnostic Deviation - Blood | Very unlikely vaccine related |
| 20012 | Extremity (gait/ambulation) lameness | Very unlikely vaccine related |

TABLE 18

AE as function of Vaccine and Booster during first two years

| | | | | | | | | |
|---|---|---|---|---|---|---|---|---|
| Month | 1 | | 2 | | 3-11 m | 12 | 13-23 m | 24 |
| Vaccines | DNA #1 | DNA #2 | PEP #1 | PEP #2 | | PEP #3 | | PEP #4 |
| # Dogs | 588 | 577 | 561 | 543 | | 291 | | |
| # AEs | 170 | 116 | 164 | 184 | | 351 | | |
| # AEs^{∗∗} | 80 (14%) | 50 (9%) | 124 (22%) | 66 (12%) | | 240 | | |

^{∗∗}AEs classified as “unknown”, “somewhat likely”, or “very likely” vaccine associated Primary toxicities: GI-Related, Injection site reaction, Lethargy

TABLE 19

AE Counts

| TOTAL # and TYPE of AE | Count |
|---|---|
| Acidosis | 5 |
| Albumin (low) | 3 |
| Alkaline phosphatase | 22 |
| Allergy reaction/Hypersensitivity | 1 |
| Alopecia | 2 |
| ALT | 27 |
| Anorexia | 37 |
| Arthritis (non-septic) | 3 |
| AST | 8 |
| Bilirubin | 1 |
| Bronchospasm wheezing | 1 |
| BUN | 3 |
| Calcium (high) | 1 |
| Calcium (low) | 1 |
| Conjunctivitis/ocular surface disease | 4 |
| Cough | 15 |
| Creatinine | 11 |
| Cystitis (haematuria/pyuria included) | 7 |
| Dermatitis | 16 |
| Diagnostic Deviation - Blood | 106 |
| Diagnostic Deviation - Urine | 53 |
| Diarrhea | 85 |
| Ear pain external ear inflammation | 8 |
| External ear pain | 1 |
| Extremity (gait/ambulation) lameness | 48 |
| Fever | 6 |
| Glucose (high) | 1 |
| Glucose (low) | 2 |
| Incontinence (urinary) | 3 |
| Injection site reaction | 59 |
| Join effusion | 1 |
| Keratitis (corneal inflammation/corneal ulceration) | 3 |
| Lethargy/fatigue/general performance | 67 |
| Nausea/ptyalism | 13 |
| Neoplasms benign malignant and unspecified (including cysts and polyps) | 1 |
| Neuropathy: cranial nerve (please specify) | 2 |
| Neuropathy: motor | 1 |
| Neutropenia | 6 |
| Neutrophilia | 1 |
| Other - please specify | 37 |
| Packed cell volume | 3 |
| Pain | 44 |
| Pancreatitis | 1 |
| Personality/behavior | 6 |
| Pollakiuria | 1 |
| Polydipsia | 19 |
| Polyuria | 13 |
| Potassium (high) | 1 |
| Potassium (low) | 1 |
| Premature ventricular contractions | 2 |
| Proteinuria | 13 |
| Prothrombin time | 1 |
| Pruritus | 22 |
| Rash: acne/acneiform | 1 |
| Seizure | 5 |
| Skin ulceration | 2 |
| Sneezing/Reverse Sneezing | 12 |
| Stranguria | 1 |
| Tachypnea (not panting) | 2 |
| Thrombocytopenia | 15 |
| Thyroid function (hypothyroidism) | 1 |
| Tremor | 2 |
| Urticarial (hives, welts, wheals) | 1 |
| Uveitis | 1 |
| Vaginal discharge (non-infectious) | 2 |
| Valvular heart disease | 2 |
| Vertigo | 2 |
| Vomiting | 135 |
| Weight loss | 3 |
| Grand Total | 986 |

Initial attrition was high, but population cohort had a high incidence of pre-existing cancer. Dose limiting GI toxicities were reconsidered. Requirements for adverse events included GI signs persisted beyond 24 hrs.

TABLE 20

Watch list AEs for future consideration

| Progressive liver enzyme elevations | (ALP 22, ALT 27, AST 8) |
|---|---|
| Lameness | 45 |
| Dermatitis | 16 |
| Creatinine elevation | 11 |
| PU | 13 |
| PD | 19 |
| Proteinuria | 13 |
| Cough | 15 |
| Thrombocytopenia | 15 |

Protocol Requirements for Adverse Events (from the final protocol): A DLT is defined as any non-hematologic Grade 3 or higher toxicity. The exceptions that are not dose limiting are: abnormalities clearly not related to study drug, such as physical injury, congenital abnormality; anorexia, vomiting or diarrhea remediable within 24 hours by medical therapy; and injection site toxicities that are significant and/or extensive but that may not meet Grade 3 criteria. A hematologic DLT is defined as an uncomplicated (e.g., no sepsis or bleeding) Grade 4 neutropenia or thrombocytopenia. If sepsis, bleeding, or another complication occurs, the toxicity should be graded according to the VCOG-CTCAE v1.1 and would be considered DLT if Grade 3 or 4.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

What is claimed is:

1. A canine cancer vaccine comprising: one or more peptides having the sequence according to one of SEQ ID NOs: 1-34 and/or a nucleic acid capable of expressing the one or more peptides and a pharmaceutically acceptable carrier.

2. The vaccine of claim 1, further comprising an adjuvant.

3. The vaccine of claim 1, further comprising a nucleic acid capable of expressing canine GMCSF.

4. The vaccine of claim 3, wherein canine GMCSF has the amino acid sequence according to SEQ ID NO: 39.

5. The vaccine of claim 3, wherein the nucleic acid capable of expressing canine GMCSF is the vector NTC9382R-MCS-GMCSF.

6. The vaccine of claim 1, wherein the vaccine comprises or more vectors expressing the peptide according to SEQ ID NOs: 1-34.

7. The vaccine of claim 6, wherein the vaccine comprises a first vector capable of expressing a peptide including the amino acid sequences according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 26, and a second vector capable of expressing a peptide including the amino acid sequences according to SEQ ID NO: 23, SEQ ID NO: 24, SEQ ID NO: 25, SEQ ID NO: 27, SEQ ID NO: 28, SEQ ID NO: 29, SEQ ID NO: 30, SEQ ID NO: 31, SEQ ID NO: 32, SEQ ID NO: 33, and SEQ ID NO: 34.

8. The vaccine of claim 7, wherein the amino acid sequences are separated by a peptide linker.

9. The vaccine of claim 8, wherein the linker comprises an amino acid sequence according to SEQ ID NO: 36.

10. The vaccine of claim 7, wherein the first vector is NTC9382R-MCS-VACCS I and the second vector is NTC9382R-MCS-VACCS II.

11. The vaccine of claim 7, further comprising a peptide component, the peptide component comprising isolated peptides with the amino acid sequences according to SEQ ID NO: 1, SEQ ID NO: 2, and SEQ ID NO: 4, SEQ ID NO: 5, SEQ ID NO: 6, and SEQ ID NO: 11, SEQ ID NO: 13, SEQ ID NO: 14, and SEQ ID NO: 15, SEQ ID NO: 17, SEQ ID NO: 20, and SEQ ID NO: 21, SEQ ID NO: 22, SEQ ID NO: 23, SEQ ID NO: 26, SEQ ID NO: 28, and SEQ ID NO: 29, SEQ ID NO: 30, SEQ ID NO: 32, SEQ ID NO: 33, and SEQ ID NO: 34.

12. A method of preventing, treating, or inhibiting cancer in a canine subject, comprising administering to the canine subject a vaccine comprising: one or more peptides having the sequence according to one of SEQ ID NOs: 1-34 and/or a nucleic acid capable of expressing the one or more peptides and a pharmaceutically acceptable carrier.

13. A method of eliciting an immune response in a canine subject, comprising administering to the canine subject a vaccine comprising: one or more peptides having the sequence according to one of SEQ ID NOs: 1-34 and/or a nucleic acid capable of expressing the one or more peptides and a pharmaceutically acceptable carrier.

14. A composition comprising: one or more peptides having the sequence according to one of SEQ ID NOs: 1-34 and/or a nucleic acid capable of expressing the one or more peptides.

15. The composition of claim 14, wherein the composition comprises one or more vectors expressing the peptide according to SEQ ID NOs: 1-34.

16. The composition of claim 15, comprising a first vector capable of expressing a peptide including the amino acid sequences according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 26, and a second vector capable of expressing a peptide including the amino acid sequences according to SEQ ID NO: 23, SEQ ID NO: 24, SEQ ID NO: 25, SEQ ID NO: 27, SEQ ID NO: 28, SEQ ID NO: 29, SEQ ID NO: 30, SEQ ID NO: 31, SEQ ID NO: 32, SEQ ID NO: 33, and SEQ ID NO: 34.

17. The composition of claim 16, wherein the amino acid sequences are separated by a peptide linker.

18. The composition of claim 17, wherein the linker comprises an amino acid sequence according to SEQ ID NO: 36.

19. The composition of claim 16, wherein the first vector is NTC9382R-MCS-VACCS I (SEQ ID NO: 37) and the second vector is NTC9382R-MCS-VACCS II (SEQ ID NO: 38).

20. A kit comprising the vaccine of claim 1 and instructions for use.


SEQUENCE LISTING (39 sequences)

SEQ ID NO: 1 - protein, 27 aa, synthetic construct
  REGION 1..27: note = Artificially Synthesized Peptide
TAIIGPNGSGCCRIYCHEEPQREDSSI

SEQ ID NO: 2 - protein, 21 aa, synthetic construct
  REGION 1..21: note = Artificially Synthesized Peptide
IPRMQPQASANPCQLLKPMVA

SEQ ID NO: 3 - protein, 21 aa, synthetic construct
  REGION 1..21: note = Artificially Synthesized Peptide
AVLLMCQMYQPWMCEEYYRLL

SEQ ID NO: 4 - protein, 30 aa, synthetic construct
  REGION 1..30: note = Artificially Synthesized Peptide
GRVIECDVVKGSCQDGEAVHRKPSPGGYRA

SEQ ID NO: 5 - protein, 30 aa, synthetic construct
  REGION 1..30: note = Artificially Synthesized Peptide
GEAVHRKPSPGGYRAGDSLTLRAVWEGAGM

SEQ ID NO: 6 - protein, 30 aa, synthetic construct
  REGION 1..30: note = Artificially Synthesized Peptide
QSRIPSKKCSLRKSTKSYKSCTSLKSLVPT

SEQ ID NO: 7 - protein, 23 aa, synthetic construct
  REGION 1..23: note = Artificially Synthesized Peptide
LLCVVSGKFVVSRSTFRHTGCYS

SEQ ID NO: 8 - protein, 12 aa, synthetic construct
  REGION 1..12: note = Artificially Synthesized Peptide
ILLNLSSESCTF

SEQ ID NO: 9 - protein, 30 aa, synthetic construct
  REGION 1..30: note = Artificially Synthesized Peptide
QWSSVTSLEWTAQERGCSSWLMKRTWMKSW

SEQ ID NO: 10 - protein, 30 aa, synthetic construct
  REGION 1..30: note = Artificially Synthesized Peptide
MKSWSLRDPSCRSTQEYISTQALWTPISTA

SEQ ID NO: 11 - protein, 30 aa, synthetic construct
  REGION 1..30: note = Artificially Synthesized Peptide
SPVASRGWVDTLKTPPSTTTGCPSWSPSWG

SEQ ID NO: 12 - protein, 30 aa, synthetic construct
  REGION 1..30: note = Artificially Synthesized Peptide
FLACVWTHSSSVSWKTWRGTTARRTGPTTC

SEQ ID NO: 13 - protein, 29 aa, synthetic construct
  REGION 1..29: note = Artificially Synthesized Peptide
SSAVAVGGPTPRIGPTPSRSSSPTGRAWS

SEQ ID NO: 14 - protein, 30 aa, synthetic construct
  REGION 1..30: note = Artificially Synthesized Peptide
NLDLQHQKKRKKKKDKVETQNRSQRANSTE

SEQ ID NO: 15 - protein, 30 aa, synthetic construct
  REGION 1..30: note = Artificially Synthesized Peptide
KMGKRKSLKRRKLQELPKRRKLRKNSNLSH

SEQ ID NO: 16 - protein, 30 aa, synthetic construct
  REGION 1..30: note = Artificially Synthesized Peptide
SHMMIKFFWMEMRYLCGSMTQFTLKHLSWD

SEQ ID NO: 17 - protein, 25 aa, synthetic construct
  REGION 1..25: note = Artificially Synthesized Peptide
NSPNGSRKRKKSCNFRINHGGLEPN

SEQ ID NO: 18 - protein, 28 aa, synthetic construct
  REGION 1..28: note = Artificially Synthesized Peptide
TQLHFAAESIIIIQLWKALAHSSCHLGA

SEQ ID NO: 19 - protein, 23 aa, synthetic construct
  REGION 1..23: note = Artificially Synthesized Peptide
KEKYRSRFYSKCLISIRSWIKTT

SEQ ID NO: 20 - protein, 30 aa, synthetic construct
  REGION 1..30: note = Artificially Synthesized Peptide
NLRRMVRGFIPCWIGTYIYLPKRKNLSYKR

SEQ ID NO: 21 - protein, 30 aa, synthetic construct
  REGION 1..30: note = Artificially Synthesized Peptide
QTSRWTRKGTISLSPLLIMFIKFRKFRSPR

SEQ ID NO: 22 - protein, 30 aa, synthetic construct
  REGION 1..30: note = Artificially Synthesized Peptide
ISPRVPCSLQKTMHLKYGLFSTFCLRTSIH

SEQ ID NO: 23 - protein, 30 aa, synthetic construct
  REGION 1..30: note = Artificially Synthesized Peptide
PRRRLLRWATYMSTSPTPLPRCATTPSTCA

SEQ ID NO: 24 - protein, 30 aa, synthetic construct
  REGION 1..30: note = Artificially Synthesized Peptide
WTLLASRCNATGFLRMVEVVEVEVEVGADQ

SEQ ID NO: 25 - protein, 30 aa, synthetic construct
  REGION 1..30: note = Artificially Synthesized Peptide
LTAKFKMGLKVFVSSTPEKADRVARLLLNL

SEQ ID NO: 26 - protein, 32 aa, synthetic construct
  REGION 1..32: note = Artificially Synthesized Peptide
MMRTKRLNKMASNQKIKLIRPRPKFRLASVDT

SEQ ID NO: 27 - protein, 30 aa, synthetic construct
  REGION 1..30: note = Artificially Synthesized Peptide
GWELGSVERMVRLQSRVRAFADSGVHSATP

SEQ ID NO: 28 - protein, 30 aa, synthetic construct
  REGION 1..30: note = Artificially Synthesized Peptide
YLAYREEGEVPELAVHGLAGDAVPRPQTAG

SEQ ID NO: 29 - protein, 30 aa, synthetic construct
  REGION 1..30: note = Artificially Synthesized Peptide
SSRESWEPGGKQQWFHKVPGSELVAPCWRL

SEQ ID NO: 30 - protein, 30 aa, synthetic construct
  REGION 1..30: note = Artificially Synthesized Peptide
RDRDGDHGARQAPRVGSQHLCAGTEHYGRR

SEQ ID NO: 31 - protein, 30 aa, synthetic construct
  REGION 1..30: note = Artificially Synthesized Peptide
GLGLFLEIFWPADPVPREWDSHREVAWPSR

SEQ ID NO: 32 - protein, 30 aa, synthetic construct
  REGION 1..30: note = Artificially Synthesized Peptide
PAGDVAGVVVGHGVRHPHRPPGLPQVWKGK

SEQ ID NO: 33 - protein, 30 aa, synthetic construct
  REGION 1..30: note = Artificially Synthesized Peptide
PPGGRGGLPPSPLLRVLYETWEDASSSPGN

SEQ ID NO: 34 - protein, 30 aa, synthetic construct
  REGION 1..30: note = Artificially Synthesized Peptide
FIARTISGITSRPMTPTKRPFTVLSAVRIT

SEQ ID NO: 35 - protein, 30 aa, synthetic construct
  REGION 1..30: note = Artificially Synthesized Peptide
DLNQRGTELRSPSVDLNKPGRHSEPAAATG

SEQ ID NO: 36 - protein, 10 aa, synthetic construct
  REGION 1..10: note = Artificially Synthesized Peptide
GSGSGSGSGS

SEQ ID NO: 37 - protein, 989 aa, synthetic construct
  REGION 1..989: note = Artificially Synthesized Peptide
MPSSVSWGILLLAGLCCLVPVSLAEDLNQRGTELRSPSVDLNKPGRHSEPAAATGDLAPQMLRELQETNAALQDVRELLRQQVKEITFLKNTVMECDACGDDPGGSILMQYIKANSKFIGITELKKLGGSNDIFNNFTVSFWLRVPKVSASHLEQYGGGSGDLTAIIGPNGSGCCRIYCHEEPQREDSSIGSGSGSGSGSIPRMQPQASANPCQLLKPMVAGSGSGSGSGSAVLLMCQMYQPWMCEEYYRLLGSGSGSGSGSGRVIECDVVKGSCQDGEAVHRKPSPGGYRAGDSLTLRAVWEGAGMGSGSGSGSGSQSRIPSKKCSLRKSTKSYKSCTSLKSLVPTGSGSGSGSGSLLCVVSGKFVVSRSTFRHTGCYSGSGSGSGSGSILLNLSSESCTFGSGSGSGSGSQWSSVTSLEWTAQERGCSSWLMKRTWMKSWSLRDPSCRSTQEYISTQALWTPISTAGSGSGSGSGSSPVASRGWVDTLKTPPSTTTGCPSWSPSWGPMSSPAASSVFLACVWTHSSSVSWKTWRGTTARRTGPTTCPKPFSRSGSGSGSGSGSSSAVAVGGPTPRIGPTPSRSSSPTGRAWSGSGSGSGSGSNLDLQHQKKRKKKKDKVETQNRSQRANSTEHQGEHNRAVGSGSGSGSGSKMGKRKSLKRRKLQELPKRRKLRKNSNLSHMMIKFFWMEMRYLCGSMTQFTLKHLSWDGSGSGSGSGSNSPNGSRKRKKSCNFRINHGGLEPNGSGSGSGSGSTQLHFAAESIIIIQLWKALAHSSCHLGAGSGSGSGSGSKEKYRSRFYSKCLISIRSWIKTTGSGSGSGSGSNLRRMVRGFIPCWIGTYIYLPKRKNLSYKRQTSRWTRKGTISLSPLLIMFIKFRKFRSPRSSTLWSQCWPFFIASSFPTVGSGSGSGSGSISPRVPCSLQKTMHLKYGLFSTFCLRTSIHGSGSGSGSGSMMRTKRLNKMASNQKIKLIRPRPKFRLASVDT

SEQ ID NO: 38 - protein, 972 aa, synthetic construct
  REGION 1..972: note = Artificially Synthesized Peptide
MPSSVSWGILLLAGLCCLVPVSLAEDLNQRGTELRSPSVDLNKPGRHSEPAAATGDLAPQMLRELQETNAALQDVRELLRQQVKEITFLKNTVMECDACGDDPGGSILMQYIKANSKFIGITELKKLGGSNDIFNNFTVSFWLRVPKVSASHLEQYGGGSGDLLLSLLLQASLPMCCDGAGRSCPLEASGKKVWTLLASRCNATGFLRMVEVVEVEVEVGADQLFPPRRRLLRWATYMSTSPTPLPRCATTPSTSLVRRKRWLFPQSRRQGAQNEGVLGHNHQEGSGSGSGSGSLTAKFKMGLKVFVSSTPEKADRVARLLLNLNQKMKSNWPGSGSGSGSGSGWELGSVERMVRLQSRVRAFADSGVHSATPKERGQILGSGSGSGSGSYLAYREEGEVPELAVHGLAGDAVPRPQTAGHPDPRKPGKRAHLLLPGSGSGSGSGSSSRESWEPGGKQQWFHKVPGSELVAPCWRLGAVSDPALQGSGSGSGSGSRDRDGDHGARQAPRVGSQHLCAGTEHYGRREECRCHGLGECAVEQTLPRYGVPRPGQPRGSGSGSGSGSGLGLFLEIFWPADPVPREWDSHREVAWPSRRGLTDHFELWRWSRREQDTDIPSHGPGGSGSGSGSGSQAALQKDQLPTILLCLQPAGDVAGVVVGHGVRHPHRPPGLPQVWKGKCHCGSEPQVGSGSGSGSGSDHQTPLVLYQEHSCRQHPAPPVTVLTELQERTLLQSSLWRISYKSLGSGSGSGSGSGLSHTAGEAAARTPRPGCKGCPPGGRGGLPPSPLLRVLYETWEDASSSPGNGSGSGSGSGSEGNWERQSPVWLLSQHGMVYLHSGHGCLLWYSRLRDDKGSGSGSGSGSAPSNPSPTTLRVEGWRCTSWEASVTTGSCHKNLLTSFLVNLTDKRRTFTGSGSGSGSGSWPPTQPRNPTSVCTVIRCFIARTICGITCRPMTPTKRPFTVLSAVRITIRS

SEQ ID NO: 39 - protein, 144 aa, synthetic construct
  REGION 1..144: note = Artificially Synthesized Peptide
MWLQNLLFLGTVVCSISAPTRSPTLVTRPSQHVDAIQEALSLLNNSNDVTAVMNKAVKVVSEVFDPEGPTCLETRLQLYKEGLQGSLTSLKNPLTMMANHYKQHCPPTPESPCATQNINFKSFKENLKDFLFNIPFDCWKPVKK